(19) United States
(12) Patent Application Publication
(10) Pub. No.: US 2018/0265894 A1
(43) Pub. Date: Sep. 20, 2018

(54) Gene Expression Cassette And Product Thereof

(71) Applicant: NATIONAL UNIVERSITY CORPORATION OKAYAMA UNIVERSITY, Okayama (JP)

(72) Inventors: Masakiyo Sakaguchi, Okayama (JP); Masahiro Nishibori, Okayama (JP); Hiromi Kumon, Okayama (JP); Hitoshi Murata, Okayama (JP); Kenichi Yamamoto, Okayama (JP); Rie Kinoshita, Okayama (JP)

(21) Appl. No.: 15/762,564

(22) PCT Filed: Oct. 3, 2016
(86) PCT No.: PCT/JP2016/079219

§ 371 Date: Mar. 23, 2018

(30) Foreign Application Priority Data
Oct. 6, 2015 (JP) 2015-198160
Mar. 23, 2016 (JP) 2016-059297

(51) Int. Cl. C12N 15/86; C07K 14/475; C07K 14/705; C07K 14/435; C12N 9/12; A61P 31/02; A61P 39/06

(52) U.S. Cl. CPC C12N 15/86; C07K 14/475; C07K 14/70521; C07K 14/70503; C07K 14/435; C12N 9/12; C07K 2319/30; A61P 31/02; A61P 39/06; C12N 2800/90; C12N 2820/00; C12N 2830/00; C12Y 207/10001

(57) ABSTRACT

Provided is a gene expression cassette for stably and highly producing a protein of interest. The gene expression cassette has a structure in which a DNA construct (X) containing a gene of interest and a poly A addition sequence is sandwiched between a promoter (P) and an enhancer (P′), the gene expression cassette further including transposon sequences (T) upstream of the promoter (P) and downstream of the enhancer (P′). Further, in the gene expression cassette, when a nuclear matrix binding sequence (M) is appropriately arranged upstream of a replication initiation sequence (S) in combination with the transposon sequence (T), the protein of interest can be more effectively produced stably and in a large amount. For example, HRG, PD-1, EMMPRIN, NPTNβ, EMB, RAGE, MCAM, ALCAM, ErbB2, and an antibody can each be produced stably and in a large amount.

### TECHNICAL FIELD

The present invention relates to a gene expression cassette capable of producing a recombinant protein in a large amount and stably, and to a method of expressing a gene including using the gene expression cassette and a product thereof. Specifically, the present invention relates to a gene expression cassette including a promoter, an enhancer, and the like, and to a method of elevating expression of a gene including using the promoter, the enhancer, and the like.

The present application claims priority from Japanese Patent Application No. 2015-198160 and Japanese Patent Application No. 2016-059297, which are incorporated herein by reference.

### BACKGROUND ART

In order to increase gene expression efficiency, various gene expression promoters, such as a CMV promoter and a CAG promoter, have been developed (Patent Literatures 1 to 3). One of such promoter systems is a system developed by the inventors of the present invention, in which a combination of a promoter, an enhancer, and the like is optimized to elevate expression of a gene in most mammalian cells (see Patent Literature 4, and Non Patent Literatures 1 and 2).

By attempting to develop a system capable of allowing a gene to be expressed with higher efficiency, and comparing and investigating promoter activities of combinations of promoters and enhancers of various genes, it has been found and reported by the inventors of the present invention that a gene can be expressed with high efficiency by using a gene expression cassette in which a DNA construct containing the gene to be expressed (hereinafter sometimes referred to as “gene of interest”) and a poly A addition sequence is linked downstream of a promoter and an enhancer or a second promoter is linked downstream of the DNA construct (see Patent Literature 4, and Non Patent Literatures 1 and 2). However, a vector containing the gene expression cassette is a vector effective for transient expression in cells. Currently, there is a need to further improve the vector in order to generate mammalian cells that stably and highly produce a protein of interest demanded in a site of pharmaceutical product production.

In general, in order to acquire cells that stably and highly produce a protein of interest by a gene recombination technique, the gene of interest is incorporated into chromosomes in host cells, and gene amplification is utilized to construct cells into which multiple copies of the gene of interest have been incorporated. The most frequently used method therefor is a method using dihydrofolate reductase (DHFR)-deficient line CHO-DG44 cells. A specific example thereof is a method involving introducing a vector containing a gene of interest and a DHFR gene into cells to acquire cells into which multiple copies of the gene of interest have been incorporated (see Non Patent Literatures 3 and 4). However, such method using DHFR-deficient cells has, for example, the following problems. Culture needs to be performed while gradually increasing a concentration of methotrexate (MTX) serving as a DHFR inhibitor, and a MTX-resistant clone at a high concentration is selected. Accordingly, acquisition of the clone takes time. In addition, versatility to other cells is low.

As described above, progress has been made in improvement of technology for increasing gene expression efficiency through development of various promoters and the like. However, it is difficult for a protein production system in mammalian cells to provide a sufficient amount of a protein as compared to other hosts, such as Escherichia coli and yeast. In the field of biotechnology, even when the above-mentioned related-art technology is used, a problem commonly occurs in that gene expression hardly occurs or an amount of an expressed protein is extremely small depending on the kind of cells and the kind of gene. In addition, this problem poses a significant obstacle in development of medicine involving using gene expression for diagnosis or treatment.

For example, a histidine-rich glycoprotein (HRG) is a plasma protein having a molecular weight of about 80 kDa identified by Heimburger et al (1972) in 1972. HRG is a high histidine-containing protein constituted of a total of 507 amino acids including 66 histidines, is mainly synthesized in the liver, and is present in human plasma at a concentration of from about 100 μg/ml to about 150 μg/ml, which is considered to be extremely high. However, in order to investigate clinical significance of HRG, a method of producing a sufficient amount of HRG by a gene recombination technology has been desired.

In addition, proteins such as programmed cell death 1 (PD-1), extracellular matrix metalloproteinase inducer (EMMPRIN), neuroplastin-β (NPTNβ), embigin (EMB), receptor for advanced glycation end products (RAGE), melanoma cell adhesion molecule (MCAM), activated leukocyte cell adhesion molecule (ALCAM), and receptor tyrosine-protein kinase erbB-2 (ErbB2) are each known to suppress immune cells or be expressed in tumor cells, and those proteins are considered to be important as candidates for a protein that may be used for research and development in a medical field, a pharmaceutical, a pharmaceutical product, a diagnostic drug, or a reagent. For those proteins, a method of producing a sufficient amount of each of the proteins by a gene recombination technology is desired.

### CITATION LIST

### Patent Literature

[PTL 1] JP 2814433 B2
[PTL 2] U.S. Pat. No. 5,168,062 A
[PTL 3] U.S. Pat. No. 5,385,839 A
[PTL 4] WO 2011/062298 A1

### Non Patent Literature

[NPL 1] Sakaguchi M. et al., Mol Biotechnol. 56(7), 621-30 (2014)
[NPL 2] Watanabe M. et al., Oncol Rep. 31(3), 1089-95 (2014)
[NPL 3] Chasin L A. et al., Proc Natl Acad Sci USA. 77 (7), 4216-20 (1980)
[NPL 4] Kaufman R J. et al., Mol Cell Biol. 3(4), 699-711 (1983)

### SUMMARY OF INVENTION

### Technical Problem

It is an object of the present invention to provide a gene expression cassette for stably and highly producing a protein of interest.

### Solution to Problem

The inventors of the present invention have made extensive investigations, and as a result, have found that a protein of interest can be stably and highly produced by using a gene expression cassette having a structure in which a DNA construct (X) containing a gene of interest and a poly A addition sequence is sandwiched between a promoter (P) and an enhancer (P′), the gene expression cassette further including transposon sequences (T) upstream of the promoter (P) and downstream of the enhancer (P′). Thus, the inventors have completed the present invention.

That is, the present invention includes the following.

1. A gene expression cassette having a structure in which a DNA construct (X) containing a gene of interest and a poly A addition sequence is sandwiched between a promoter (P) and an enhancer (P′), the gene expression cassette further including transposon sequences (T) upstream of the promoter (P) and downstream of the enhancer (P′).

2. A gene expression cassette according to the above-mentioned item 1, further including a replication initiation sequence (S), which is arranged upstream of the promoter (P) and/or downstream of the enhancer (P′).

3. A gene expression cassette according to the above-mentioned item 1 or 2, wherein the gene expression cassette includes the promoter (P), the DNA construct (X) containing a gene of interest and a poly A addition sequence, the enhancer (P′), and the transposon sequences (T), and optionally further includes a replication initiation sequence (S) in any one of the following orders 1) to 4):

### 1) (T), (P), (X), (P′), (T);

### 2) (T), (S), (P), (X), (P′), (T);

### 3) (T), (P), (X), (P′), (S), (T); and

### 4) (T), (S), (P), (X), (P′), (S), (T).

4. A gene expression cassette according to the above-mentioned item 2 or 3, further including a nuclear matrix binding sequence (M), which is arranged upstream of the replication initiation sequence (S).

5. A gene expression cassette, including:

a transposon sequence (T);

a promoter (P);

a DNA construct (X) containing a gene of interest and a poly A addition sequence;

an enhancer (P′);

a nuclear matrix binding sequence (M);

a replication initiation sequence (S); and

another transposon sequence (T).

6. A gene expression cassette according to any one of the above-mentioned items 2 to 5, wherein the sequence (S) includes ROIS and/or ARS.

7. A gene expression cassette according to any one of the above-mentioned items 1 to 6, wherein the promoter (P) includes a promoter selected from the group consisting of a CMV promoter, a CMV-i promoter, an SV40 promoter, an hTERT promoter, a β-actin promoter, and a CAG promoter.

8. A gene expression cassette according to any one of the above-mentioned items 1 to 7, wherein the enhancer (P′), which is linked downstream of the DNA construct (X) containing a gene of interest and a poly A addition sequence, contains any one kind or a plurality of kinds selected from an hTERT enhancer, a CMV enhancer, and an SV40 enhancer.

9. A gene expression cassette according to any one of the above-mentioned items 1 to 8, wherein, in the DNA construct (X) containing a gene of interest and a poly A addition sequence, the gene of interest contains a gene encoding part or a whole of a protein selected from HRG, PD-1, EMMPRIN, NPTNβ, EMB, RAGE, MCAM, ALCAM, ErbB2, and an antibody.

10. A gene expression plasmid, including the gene expression cassette of any one of the above-mentioned items 1 to 9.

11. A gene expression vector, including the gene expression cassette of any one of the above-mentioned items 1 to 9.

12. A method of expressing a gene of interest, including using the expression cassette of any one of the above-mentioned items 1 to 9.

13. A protein, which is produced using the gene expression cassette of any one of the above-mentioned items 1 to 9.

### Advantageous Effects of Invention

According to the present invention, a gene expression cassette suited for transient expression (e.g., a pCMViR-TSC vector described in Patent Literature 4) is sandwiched between the transposon sequences (T), and thus a large number of copies of the gene expression cassette can be inserted into chromosomes with high efficiency. When the replication initiation sequence (S) and the nuclear matrix binding sequence (M) are further linked upstream or downstream, or upstream and downstream of the gene expression cassette, the number of copies of the gene expression cassette can be amplified with high efficiency. Specifically, cells that stably and highly produce the protein of interest are obtained by linking: the transposon sequence (T) upstream of the promoter (P); the DNA construct (X) containing a gene of interest and a poly A addition sequence downstream of the promoter (P); the enhancer (P′) downstream of the DNA construct (X); and “the nuclear matrix binding sequence (M), the replication initiation sequence (S), and the transposon sequence (T) downstream of the enhancer (P′). The novel gene expression vector of the present invention has achieved an expression amount surpassing even the transient expression amount of the pCMViR-TSC vector, which has achieved production several times to several tens times as high as that achieved by a related-art gene expression vector, even in a stably expressing cell line after drug selection.

### BRIEF DESCRIPTION OF DRAWINGS

FIG. 1(A) is a diagram for illustrating the structure of a construct of an expression vector pCMViR-TSC capable of producing a protein with the highest efficiency in transient expression. FIG. 1(A) is a diagram for illustrating a gene expression plasmid vector obtained by inserting a gene expression cassette for transient expression into a promoter-less cloning plasmid vector from IDT Inc. (pIDT-SMART). FIG. 1(B) is a diagram for illustrating an example of a gene expression plasmid vector of the present invention constructed using pIDT-SMART of FIG. 1(A) as a backbone (Example 1).

FIG. 2 is a conceptual diagram for illustrating the configurations of various gene expression cassettes No. 1 to No. 10 specifically constructed in Examples (Examples 1 and 2).

FIG. 3 is a diagram for illustrating the base sequence of 5′-side TP (5′TP) upstream of a promoter (P) (SEQ ID NO: 1) and the complete sequence of a 5′-side TP region (SEQ ID NO: 2) (Example 1).

FIG. 4 is a diagram for illustrating the base sequence of 3′-side TP (3′TP) downstream of an enhancer (P′) (SEQ ID NO: 3) and the complete sequence of a 3′-side TP region (SEQ ID NO: 4) (Example 1).

FIG. 5-1 is a diagram for illustrating part of the complete base sequence of a No. 4 gene expression vector (part of SEQ ID NO: 8) (Example 2).

FIG. 5-2 is a diagram for illustrating part of the complete base sequence of the No. 4 gene expression vector (part of SEQ ID NO: 8, continuation of FIG. 5-1) (Example 2).

FIG. 5-3 is a diagram for illustrating part of the complete base sequence of the No. 4 gene expression vector (part of SEQ ID NO: 8, continuation of FIG. 5-2) (Example 2).

FIG. 6-1 is a diagram for illustrating part of the complete base sequence of a No. 1 gene expression vector (part of SEQ ID NO: 9) (Example 2).

FIG. 6-2 is a diagram for illustrating part of the complete base sequence of the No. 1 gene expression vector (part of SEQ ID NO: 9, continuation of FIG. 6-1) (Example 2).

FIG. 7 is a graph for showing results of measurement of the GFP fluorescence intensities of cells for each of CHO cells having introduced therein gene expression vectors respectively corresponding to No. 1 to No. 10 and a control (Example 2).

FIG. 8 is a graph for showing results of measurement of the GFP fluorescence intensities of cells for each of HEK293T cells having introduced therein the gene expression vectors respectively corresponding to No. 1 to No. 10 and the control (Example 2).

FIG. 9 is a graph for showing results of correction of the GFP fluorescence intensity of cells with a protein quantitative value for each of the CHO cells having introduced therein the gene expression vectors respectively corresponding to No. 1 to No. 10 (Example 2).

FIG. 10 is a graph for showing results of correction of the GFP fluorescence intensity of cells with a protein quantitative value for each of the HEK293T cells having introduced therein the gene expression vectors respectively corresponding to No. 1 to No. 10 (Example 2).

FIG. 11 is a diagram for illustrating the structure of an HRG-carrying construct for generating HRG (No. 4-HRG) (Example 3).

FIG. 12 is a graph for showing results of confirmation of an influence on the morphology of neutrophils when HRG is added to a neutrophil culture system for HRG generated using a No. 4-HRG gene expression cassette-containing vector (Experimental Example 3-1).

FIG. 13 is a graph for showing results of confirmation of an influence on the survival rate of CLP sepsis model mice for the HRG generated using the No. 4-HRG gene expression cassette-containing vector (Experimental Example 3-2).

FIG. 14 is a graph for showing results of confirmation of an influence on production-suppressing activity on a reactive oxygen molecular species for the HRG generated using the No. 4-HRG gene expression cassette-containing vector (Experimental Example 3-3).

FIG. 15 is a diagram for illustrating the amino acid sequence of a human IgG₂ Fc region (Example 4).

FIG. 16 is a diagram for illustrating the complete base sequence of the extracellular domain of PD-1 (Example 4).

FIG. 17 is a diagram for illustrating the complete base sequence of the extracellular domain of EMMPRIN (Example 5).

FIG. 18 is a diagram for illustrating the complete base sequence of the extracellular domain of NPTNβ (Example 6).

FIG. 19 is a diagram for illustrating the complete base sequence of the extracellular domain of EMB (Example 7).

FIG. 20 is a diagram for illustrating the complete base sequence of the extracellular domain of RAGE (Example 8).

FIG. 21 is a diagram for illustrating the complete base sequence of the extracellular domain of MCAM (Example 9).

FIG. 22 is a diagram for illustrating the complete base sequence of the extracellular domain of ALCAM (Example 10).

FIG. 23 is a diagram for illustrating the complete base sequence of the extracellular domain of ErbB2 (Example 11).

FIG. 24 is a diagram for illustrating the complete base sequence of HRG (Example 12).

FIG. 25 are images for showing SDS-PAGE results of respective Fc fusion proteins obtained in Examples 4 to 12 (Experimental Example 4).

FIG. 26 is a graph for showing results of confirmation of an influence on the morphology of neutrophils when HRG is added to a neutrophil culture system for each of recombinant human HRG (rHRG), HRG-Fc, and plasma-derived HRG (hHRG) based on a sphere-forming rate (%) (Experimental Example 5).

FIG. 27 are photographs for showing results of confirmation of an influence on the morphology of neutrophils when HRG is added to a neutrophil culture system for each of recombinant human HRG (rHRG), HRG-Fc, and plasma-derived HRG (hHRG) (Experimental Example 5).

FIG. 28 are graphs for showing results of confirmation of a blocking effect on the cancer cell chemotaxis-promoting action of S100A8/A9 for each of NPTN-Fc and EMMPRIN-Fc generated in Examples (Experimental Example 6).

FIG. 29 is a graph for showing results of confirmation of a blocking effect on the cancer cell chemotaxis-promoting action of S100A8/A9 for each of RAGE-Fc, ALCAM-Fc, MCAM-Fc, and EMB-Fc generated in Examples (Experimental Example 6).

### DESCRIPTION OF EMBODIMENTS

Herein, the term “gene expression cassette” refers to a DNA set for enabling a protein of interest to be expressed by a gene recombination operation, and more specifically, refers to a DNA set including a gene encoding a protein of interest (gene of interest), and further including various DNA sequences for enabling the gene to be expressed. Herein, the term “protein of interest” means a protein to be expressed and/or produced.

Herein, the “gene expression cassette” includes at least DNAs having the functions of a “promoter (P)”, a “DNA construct (X) containing a gene of interest and a poly A addition sequence”, and an “enhancer (P′)”, and further includes “transposon sequences (T)”. The “gene expression cassette” may further include a “replication initiation sequence (S)” and a “nuclear matrix binding sequence (M)”.

The “gene expression cassette” of the present invention has a structure in which the DNA construct (X) containing a gene of interest and a poly A addition sequence is sandwiched at least between the promoter (P) and the enhancer (P′), and further includes the transposon sequences (T) upstream of the promoter (P″ and downstream of the enhancer (P′). Further, the nuclear matrix binding sequence (M) and the replication initiation sequence (S) may be arranged upstream of the promoter (P) and/or downstream of the enhancer (P′).

According to the present invention, a gene expression cassette suited for transient expression (e.g., a pCMViR-TSC vector described in Patent Literature 4) is sandwiched between the transposon sequences (T), and thus a large number of copies of the gene expression cassette can be inserted into chromosomes with high efficiency. When the replication initiation sequence (S) and the nuclear matrix binding sequence (M) are further linked upstream or downstream, or upstream and downstream of the gene expression cassette, the number of copies of the gene expression cassette can be amplified with high efficiency. Specifically, cells that stably and highly produce the protein of interest are obtained by linking: the transposon sequence (T) upstream of the promoter (P); the DNA construct (X) containing a gene of interest and a poly A addition sequence downstream of the promoter (P); the enhancer (P′) downstream of the DNA construct (X); and the nuclear matrix binding sequence (M), the replication initiation sequence (S), and the transposon sequence (T) downstream of the enhancer (P′).

The “gene expression cassette” of the present invention is configured in at least any one of the following orders 1) to 4), where (P) represents a promoter, (X) represents a DNA construct containing a gene of interest and a poly A addition sequence, (P′) represents an enhancer, (T) represents a transposon sequence, and (S) represents a replication initiation sequence. Herein, the transposon sequences (T) may each contain two or more sequences each identifying a transposon that are linked to each other.

### 1) (T), (P), (X), (P′), and (T);

### 2) (T), (S), (P), (X), (P′), (T);

### 3) (T), (P), (X), (P′), (S), (T); and

### 4) (T), (S), (P), (X), (P′), (S), (T).

Herein, in the “DNA construct (X) containing a gene of interest and a poly A addition sequence,” the gene of interest contains a gene (DNA) encoding a protein of interest, and may be a gene of a different origin from host cells or may be a gene of the same origin. In addition, the DNA construct (X) may contain a reporter gene for detecting an expressed gene or diagnosing a disease. A sequence known per se may be applied as the poly A addition sequence (polyadenylation sequence, polyA), its origin is not limited, and examples thereof include growth hormone gene-derived poly A addition sequences, such as a bovine growth hormone gene-derived poly A addition sequence and a human growth hormone gene-derived poly A addition sequence, an SV40 virus-derived poly A addition sequence, and a human or rabbit β-globin gene-derived poly A addition sequence. The incorporation of the poly A addition sequence into the gene expression cassette increases transcription efficiency.

Herein, the kind of the gene of interest is not limited, and DNA encoding a protein of interest to be produced by a gene recombination technology or DNA encoding a protein of interest to be expressed in vivo for use in treatment of a specific disease may be used. Examples of the protein of interest described herein include proteins each of which may be used for research and development in a medical field, a pharmaceutical, a pharmaceutical product, a diagnostic drug, or a reagent. Such protein may be a protein known per se or a protein to be discovered in the future. The protein may be a whole protein, or may be a partial protein. Further, the protein may be formed of a complex. Examples of the protein include histidine-rich glycoprotein (HRG), programmed cell death 1 (PD-1), extracellular matrix metalloproteinase inducer (EMMPRIN), neuroplastin-3 (NPTNβ), embigin (EMB), receptor for advanced glycation end products (RAGE), melanoma cell adhesion molecule (MCAM), activated leukocyte cell adhesion molecule (ALCAM), receptor tyrosine-protein kinase erbB-2 (ErbB2), and an antibody. Further, as described later, the protein of interest may be an Fc fusion protein, which is obtained by fusing a protein with an Fc region of an antibody, or the like.

For example, HRG may be prepared by cloning, into an expression vector, full-length cDNA encoding HRG or cDNA encoding part thereof having the activity of HRG, such as full-length cDNA encoding the amino acid sequence of mature HRG (SEQ ID NO: 10) or cDNA encoding part thereof. For example, HRG may also be prepared from all or part of nucleotides identified by GenBank Accession No. NM000412 through the use of a gene recombination technology. HRG serving as an active ingredient of the present invention may be the whole of mature HRG, or may be a partial protein or peptide of mature HRG having HRG activity. Further, the HRG may contain a sugar chain or may not contain a sugar chain. HRG functions as a neutrophil activation regulator, and may be utilized as an active ingredient of a therapeutic agent for a disease caused by neutrophil activation. HRG is also effective for a method of treating a disease caused by neutrophil activation and/or an inflammatory disease accompanied by neutrophil activation.

As with the case of HRG, on the basis of full-length cDNA encoding, for example, the amino acid sequence of a mature PD-1 extracellular domain (SEQ ID NO: 14), the amino acid sequence of a mature EMMPRIN extracellular domain (SEQ ID NO: 16), the amino acid sequence of a mature NPTNβ extracellular domain (SEQ ID NO: 18), the amino acid sequence of a mature EMB extracellular domain (SEQ ID NO: 20), the amino acid sequence of a mature RAGE extracellular domain (SEQ ID NO: 22), the amino acid sequence of a mature MCAM extracellular domain (SEQ ID NO: 24), the amino acid sequence of a mature ALCAM extracellular domain (SEQ ID NO: 26), or the amino acid sequence of a mature ErbB2 extracellular domain (SEQ ID NO: 28), or cDNA encoding part thereof, each protein may be prepared through cloning into an expression vector. As a method of preparing the gene of interest in the case where the protein is fused with Fc, a method known per se or any method to be developed in the future may be applied.

PD-1 is a single-pass transmembrane protein present on an immune cell side and serving to suppress immune cells, and is disclosed in Zou W, Wolchok J D, Chen L. PD-L1 (B7-H1) and PD-1 pathway blockade for cancer therapy: Mechanisms, response biomarkers, and combinations. Sci Transl Med. 2016 Mar. 2; 8(328): 328rv4. doi: 10.1126/scitranslmed.aad7118. EMMPRIN, NPTN, EMB, RAGE, MCAM, ALCAM, and the like are present in cancer cells, and are known as adhesion molecules belonging to the immunoglobulin superfamily (single-pass transmembrane proteins). EMMPRIN is disclosed in Kanekura T, Chen X. CD147/basigin promotes progression of malignant melanoma and other cancers. J Dermatol Sci. 2010 March; 57(3): 149-54. doi: 10.1016/j.jdermsci.2009.12.008., NPTNβ is disclosed in Owczarek S, Berezin V. Neuroplastin: cell adhesion molecule and signaling receptor. Int J Biochem Cell Biol. 2012 January; 44(1): 1-5. doi: 10.1016/j.biocel.2011.10.006., EMB is disclosed in Chao F, Zhang J, Zhang Y, Liu H, Yang C, Wang J, Guo Y, Wen X, Zhang K, Huang B, Liu D, Li Y. Embigin, regulated by HOXC8, plays a suppressive role in breast tumorigenesis. Oncotarget. 2015 Sep. 15; 6(27): 23496-509, RAGE is disclosed in Sims G P, Rowe D C, Rietdijk S T, Herbst R, Coyle A J. HMGB1 and RAGE in inflammation and cancer. Annu Rev Immunol. 2010; 28: 367-88. doi: 10.1146/annurev.immunol.021908.132603., MCAM is disclosed in Wang Z, Yan X. CD146, a multi-functional molecule beyond adhesion. Cancer Lett. 2013 Apr. 28; 330(2): 150-62. doi: 10.1016/j.canlet.2012.11.049., and ALCAM is disclosed in Ofori-Acquah S F, King J A. Activated leukocyte cell adhesion molecule: a new paradox in cancer. Transl Res. 2008 March; 151(3): 122-8. doi: 10.1016/j.trsl.2007.09.006. ErbB2, which is present in cancer cells, is known as an oncogene, and its overexpression is said to be largely involved in oncogenesis and subsequent cancer progression. ErbB2 is disclosed in Appert-Collin A, Hubert P, Cremel G, Bennasroune A. Role of ErbB Receptors in Cancer Cell Migration and Invasion. Front Pharmacol. 2015 Nov. 24; 6: 283. doi: 10.3389/fphar.2015.00283.

For example, when an Fc fusion protein obtained by fusing any of the above-mentioned proteins with an Fc region of an antibody is generated, it is suitable that cDNA encoding Fc be bonded to a site encoding the C′-terminal side of the protein.

The antibody is constituted of polypeptides called heavy chains (H chains) and light chains (L chains). In addition, the H chains are each constituted of a variable region (VH) and a constant region (CH) from the N-terminal side, and the L chains are each constituted of a variable region (VL) and a constant region (CL) from the N-terminal side. CH is further constituted of the respective domains of CH1, a hinge, CH2, and CH3 from the N-terminal side. In addition, CH2 and CH3 are together called an Fc region. The antibody to be generated by the method of the present invention may be an intact antibody or an antibody fragment that is a low-molecular-weight antibody. As a class of the antibody, there are given, for example, immunoglobulin G (IgG), immunoglobulin A (IgA), immunoglobulin E (IgE), and immunoglobulin M (IgM). The antibody that may be produced by the method of the present invention is preferably IgG. As a subclass of IgG, there are given, for example, IgG1, IgG2, IgG3, and IgG4. The subclass of the antibody that may be produced by the method of the present invention may be appropriately determined depending on applications and purposes, and the antibody may be of any subclass. An example of the antibody fragment is a functional antibody fragment thereof selected from the group consisting of Fv, Fab, (Fab′)₂, Fab′, an Fc fragment, and a diabody. For example, an Fc fusion protein obtained by fusing an Fc region of an antibody with a required protein may be adopted.

A transposon (TP) is a base sequence capable of transposition between positions on a genome in a cell. The transposon is also called a jumping gene or a transposable element. Transposons are classified into a DNA type, in which a DNA fragment directly undergoes transposition, and an RNA type, which undergoes a process including transcription and reverse transcription. The transposon is also found in, for example, microorganisms, such as bacteria and yeast. Such transposable elements are DNAs having certain base sequences, and a plurality thereof are present in each cell as constituents of normal chromosomes of bacteria, yeast, and the like. This element is present by being incorporated into a chromosome, and hence is also called an insertion sequence (IS). The transposon is useful as a vector for gene introduction or as a mutagen, and is applied in various organisms in genetics and molecular biology. Herein as well, the transposon is incorporated into the gene expression cassette. Herein, the term “transposon sequence (T)” refers to a sequence identifying the transposon. As already described, the transposon sequences (T) may each contain two or more sequences each identifying a transposon that are linked to each other.

A replication reaction of DNA starts at a fixed site on chromosomes, namely an origin of replication, and proceeds in both directions. An extremely large number of origins of replication are needed in order to accurately replicate long and large chromosomal DNA of a eukaryote at a fixed time in its cell cycle, and their activities are considered to be temporally and spatially controlled. Further, various reactions occurring on chromosomes, including DNA replication, are closely associated with each other to keep chromosomal homeostasis. A region in which replication initiation occurs includes a replication initiation sequence, and examples thereof include a replication origin initiation sequence (ROIS) and an autonomously replicating sequence (ARS).

The nuclear matrix is considered not only to hold chromatin in a nucleus, but also to constitute important sites for the functioning of various intranuclear events, typified by transcription and replication of genes, DNA damage repair, and apoptosis. A plasmid containing a replication initiation sequence and a nuclear matrix binding region is considered to efficiently amplify a gene in cells. Herein, the term “nuclear matrix binding sequence (M)” refers to a sequence identified as a sequence that binds to the nuclear matrix. The gene expression cassette of the present invention more suitably includes the nuclear matrix binding sequence (M) together with the replication initiation sequence (S). In this case, the nuclear matrix binding sequence (M) is suitably arranged in a region upstream of the replication initiation sequence (S).

A promoter is a specific base sequence on DNA for initiating transcription with the DNA being a template, and is arranged upstream of the gene of interest. For a promoter that may be used in the “promoter (P)” herein, reference may be made to a “first promoter” described in Patent Literature 4, and for the “enhancer (P′)”, reference may be made to the description of an “enhancer” or a “second promoter” described in Patent Literature 4. Specific description is given below.

Herein, the sequence of the “promoter (P)” is not particularly limited, and a promoter known per se may be applied. A non-specific promoter capable of promoting the expression of the gene of interest in all kinds of cells and tissues and a specific or selective promoter, such as a tissue- or organ-specific promoter, a tumor-specific promoter, or a development- or differentiation-specific promoter, may each be used. In particular, as a promoter applicable in the present invention, a promoter that increases the number of copies of an infectious plasmid or a proliferating cell-specific promoter is suitable. Specifically, there are given, for example, an SV40 promoter, a CMV promoter, a β-actin promoter, a CAG promoter, an EF1-alpha promoter, and a ubiquitin promoter. More specifically, for example, a CMV-i promoter (hCMV+intron promoter) is used. An animal species from which the β-actin promoter is derived is not limited, and a mammalian β-actin promoter, such as a human β-actin promoter, or a chicken actin promoter is used. In addition, an artificial hybrid promoter, such as the CMV-i promoter, may also be used. The CMV-i promoter may be synthesized in accordance with the descriptions of U.S. Pat. No. 5,168,062 A and U.S. Pat. No. 5,385,839 A. In addition, depending on applications, a human telomerase reverse transcriptase (hTERT), prostate-specific antigen (PSA), c-myc, or GLUT promoter or the like may be linked as a cancer/tumor-specific promoter, a U6 or H1 promoter or the like may be linked as a promoter for expressing short hairpin RNA (shRNA) for the purpose of suppressing gene expression, an OCT3/4 or NANOG promoter or the like may be linked as an ES cell/cancer stem cell-specific promoter, a Nestin promoter or the like may be linked as a neural stem cell-specific promoter, an HSP70, HSP90, or p53 promoter or the like may be linked as a cell stress sensitive promoter, an albumin promoter or the like may be linked as a liver cell-specific promoter, and a TNF-alpha promoter or the like may be linked as a radiosensitive promoter.

Herein, the “enhancer (P′)” only needs to increase the amount of messenger RNA (mRNA) to be produced by transcription as a result, and is not particularly limited. The enhancer is a base sequence having a promoting effect on the action of a promoter, and many enhancers are generally formed of around 100 bp. The enhancer can promote transcription irrespective of the direction of a sequence. The enhancer (P′) to be used in the present invention may include one kind of enhancer, but a plurality of enhancers including two or more enhancers identical to each other may be used or a plurality of enhancers different from each other may be used in combination. Their order is not specifically limited. For example, a CMV enhancer, an SV40 enhancer, an hTERT (telomerase reverse transcriptase) enhancer, or the like may be used. As an example, the hTERT enhancer, the SV40 enhancer, and the CMV enhancer may be linked in the stated order. The enhancer (P′) is arranged downstream of the DNA construct (X) containing a gene of interest herein and a poly A addition sequence, to thereby enable gene expression to be stronger expression. For example, the enhancer (P′) may have a function similar to that of a promoter, and a sequence similar to that of a promoter. The promoter (P) and the enhancer (P′) may be identical to or different from each other. For example, a specific promoter and a non-specific promoter may be used as the promoter (P) and the enhancer (P′), respectively. Specifically, a combination of the CMV-i promoter and the CMV enhancer enables strong protein expression of the gene of interest in almost all cells (host cells) when any gene is inserted and even when any transfection reagent is used.

In the present invention, in addition to the enhancer contained in the enhancer (P′), an enhancer sequence may be arranged upstream of the promoter (P). When one or more enhancer sequences are inserted upstream of the promoter (P), the expression of a specific gene, such as a REIC/Dkk-3 gene or a CD133 gene is further enhanced in specific cells (e.g., an HEK293 cell line or an MCF7 cell line described in Examples of Patent Literature 4). In addition, when, for example, four CMV enhancers are inserted upstream of the promoter (P), further enhancement of expression is expected in specific cells (e.g., a HepG2 cell line or a HeLa cell line).

The “gene expression cassette” of the present invention may be utilized by being incorporated into a gene expression vector. The present invention also encompasses a vector containing the gene expression cassette of the present invention.

In the gene expression cassette of the present invention, a site at which the gene of interest is to be inserted may be present as a multiple cloning site. In this case, the gene of interest may be inserted at the multiple cloning site (insertion site) by utilizing a sequence to be recognized by a restriction enzyme. A gene expression cassette that does not contain DNA of the gene of interest itself but contains a portion at which the DNA is to be inserted as a multiple cloning site as described above is also encompassed in the present invention.

Further, as described in Patent Literature 4, RU5′ may be linked immediately upstream of DNA encoding the protein of interest. The term “immediately upstream” refers to direct linking without through any other element having a specific function, but a short sequence may be included as a linker between RU5′ and the DNA encoding the protein of interest. Further, SV40-ori may be linked most upstream of the gene expression cassette. SV40-ori is a binding region for an SV40 gene, and when the SV40 gene is inserted later, gene expression is elevated. Each of the above-mentioned elements needs to be functionally linked. Herein, the phrase “be functionally linked” means that each element is linked so that its function is exhibited to enhance the expression of the gene of interest.

Examples of the vector into which the gene expression cassette of the present invention is inserted include: a plasmid; viral vectors, such as an adenovirus (Ad) vector, an adeno-associated virus (AAV) vector, a lentivirus vector, a retrovirus vector, a herpes virus vector, and a Sendai virus vector; and non-viral vectors, such as a biodegradable polymer. The vector into which the gene expression cassette has been introduced may be introduced into cells by a known method, such as infection or electroporation. As the method of introducing the gene, a method known per se or any method to be developed in the future may be applied, and for example, the gene may be introduced using a known transfection reagent.

Further, a commercially available vector may be modified so as to contain the expression cassette of the present invention. For example, a commercially available vector such as a pShuttle vector may be used by incorporating an enhancer into a downstream region of its gene expression cassette.

Further, the present invention also encompasses a viral vector containing the expression cassette for the gene of interest described above. Among viral vectors, for example, an Ad vector and an AAV vector each enable specific diagnosis or treatment of a disease such as cancer, but the gene expression cassette of the present invention can allow the gene to be expressed stably and sustainably, and hence the viral vectors are desirably used as appropriate for applications of gene expression. The viral vector may be generated by inserting the expression cassette for the gene of interest described above onto a viral genome usable as a vector.

When a vector having inserted therein the gene expression cassette of the present invention is introduced into cells to transfect the cells, the gene of interest can be expressed in the cells to produce the protein of interest. A system of eukaryotic cells or prokaryotic cells may be used to introduce the gene expression cassette of the present invention to produce the protein of interest. Examples of the eukaryotic cells include cells such as established mammalian cell systems and insect cell systems, filamentous fungal cells, and yeast cells, and examples of the prokaryotic cells include bacterial cells of Escherichia coli, hay bacilli, bacteria of the genus Brevibacillus, and the like. Of those, mammalian cells, such as HEK293 cells, CHO cells, Hela cells, COS cells, BHK cells, or Vero cells, are preferably used. The protein of interest can be produced by culturing the above-mentioned host cells that have been transformed in vitro or in vivo. The culture of the host cells is performed in accordance with a known method. For example, as a culture solution, a known medium for culture, such as DMEM, MEM, RPMI1640, or IMDM, may be used. The produced protein may be purified by a known method from the culture solution in the case of a secretory protein, or from a cell extract in the case of a non-secretory protein. When proteins of interest are produced, the proteins of interest may be produced by simultaneously transfecting cells with a plurality of vectors containing different genes of interest. In this manner, a plurality of proteins can be simultaneously produced.

### EXAMPLES

The present invention is specifically described below by way of Examples, but the present invention is not limited to these Examples.

### (Example 1) Gene Expression Cassette

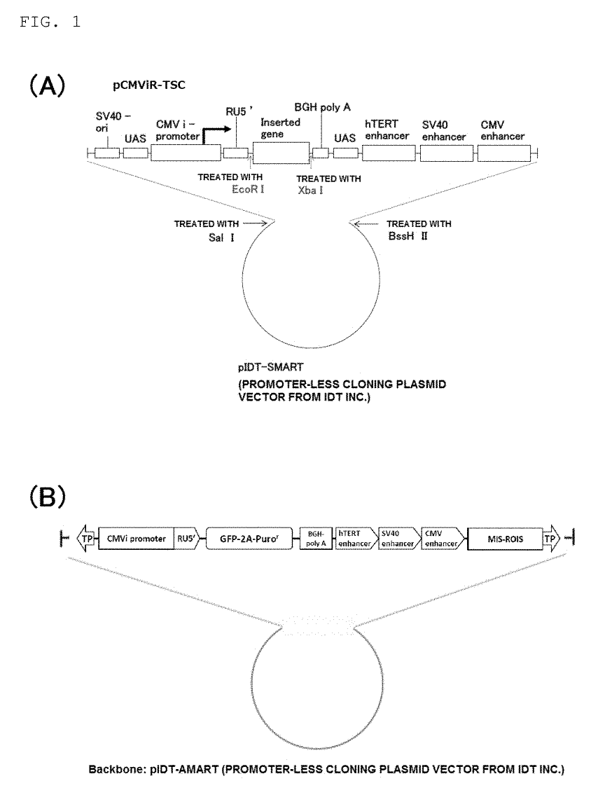

In this Example, gene expression cassettes of various constructs were constructed. Specifically, a gene expression cassette capable of producing a protein with the highest efficiency in transient expression described in Patent Literature 4 was inserted into a promoter-less cloning plasmid vector from IDT Inc. (pIDT-SMART) to generate a pCMViR-TSC expression vector (FIG. 1). pCMViR means that an RU5′ sequence is inserted downstream of a CMV-i promoter.

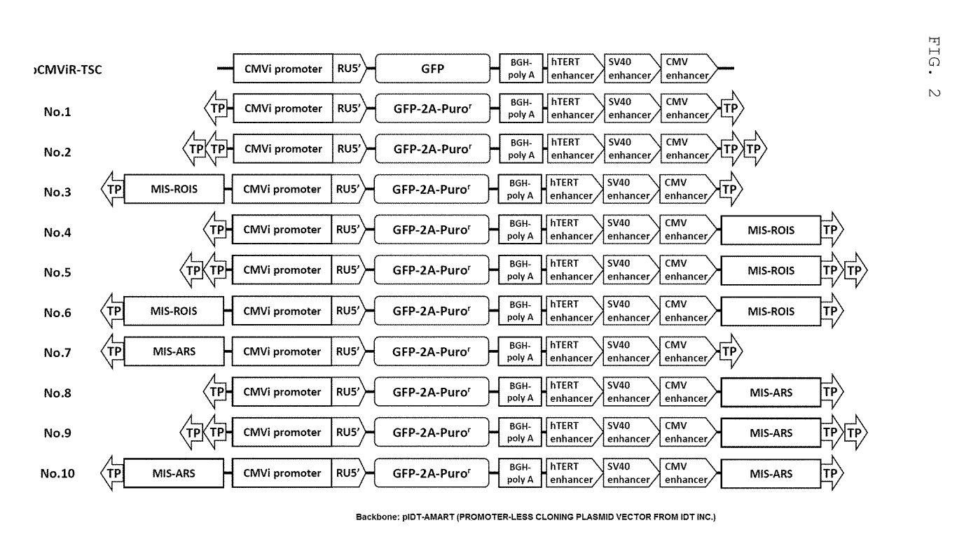

Gene expression cassettes of the present invention were constructed by linking the transposon sequences (T), the replication initiation sequence (S), and the nuclear matrix binding sequence (M) upstream and downstream of the DNA construct (X) containing a gene of interest and a poly A addition sequence according to each of the combinations of No. 1 to No. 10 illustrated in FIG. 2.

In this Example, the gene of interest contained GFP (green fluorescent protein), Puro^{r} (puromycin resistance), and 2A (2A self-processing peptide-short self-processing peptide), and was represented by “GFP-2A-Puro^{r}”. In addition, “BGH polyA” was used as the poly A addition sequence, “CMViR promoter” was used as the promoter (P), and “an hTERT enhancer, an SV40 enhancer, and a CMV enhancer linked to each other” were used as the enhancer (P′). In FIG. 2, the transposon sequences (T) are each represented by “TP”, the replication initiation sequence (S) is represented by “ROIS” or “ARS”, and the nuclear matrix binding sequence (M) is represented by “MIS”. The transposon sequence (T) may contain a plurality of sequences each identified by “TP” as illustrated in, for example, No. 2, 5, or 9.

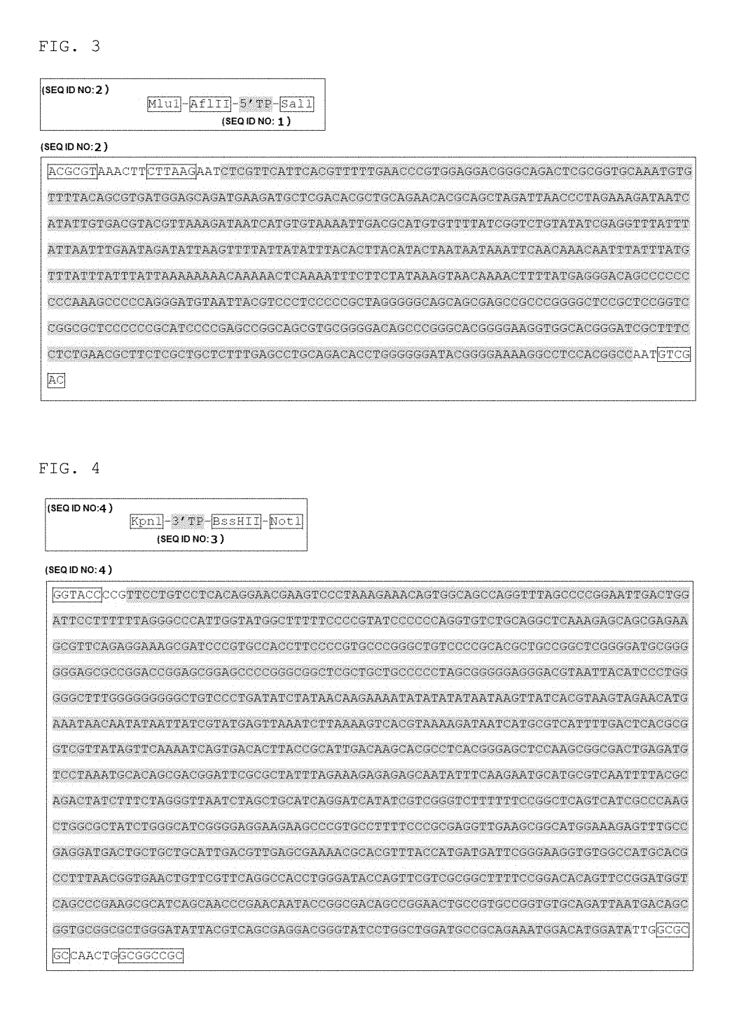

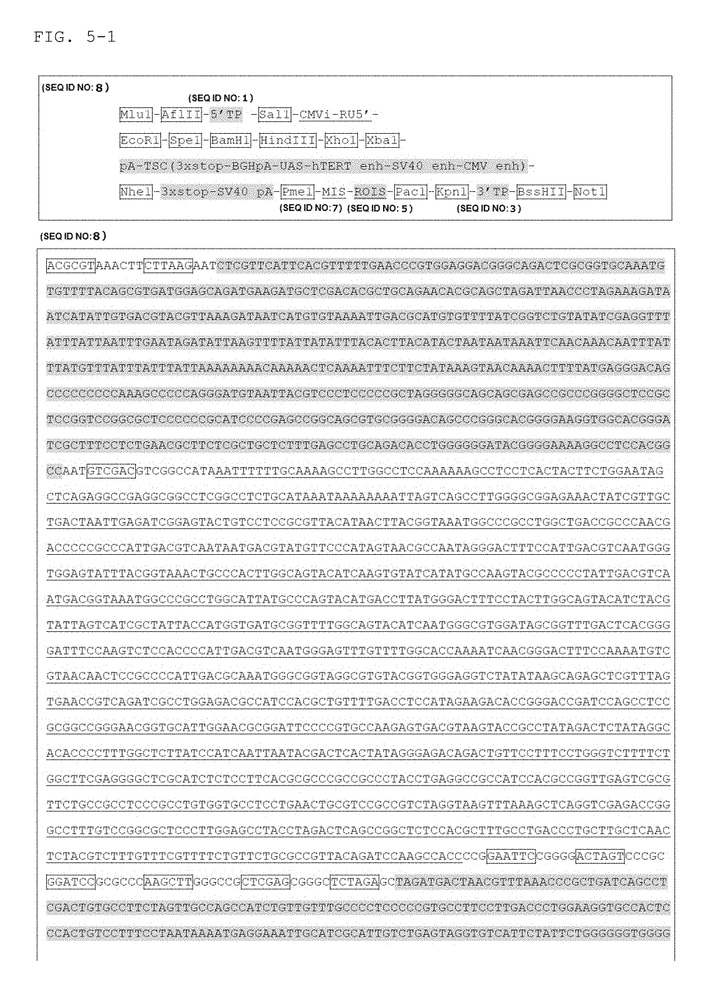

In the foregoing, the base sequence of 5′-side TP (5′TP) upstream of the promoter (P) is set forth in SEQ ID NO: 1, and the complete sequence of a 5′-side TP region is set forth in SEQ ID NO: 2 (see FIG. 3). In addition, the base sequence of 3′-side TP (3′TP) downstream of the enhancer (P′) is set forth in SEQ ID NO: 3, and the complete sequence of a 3′-side TP region is set forth in SEQ ID NO: 4 (see FIG. 4). In addition, in the replication initiation sequence (S), the base sequence of ROIS is set forth in SEQ ID NO: 5, and the base sequence of ARS is set forth in SEQ ID NO: 6. Further, the base sequence of MIS is set forth in SEQ ID NO: 7.

5′TP
(SEQ ID NO: 1)
CTCGTTCATTCACGTTTTTGAACCCGTGGAGGACGGGCAGACTCGCGGT
GCAAATGTGTTTTACAGCGTGATGGAGCAGATGAAGATGCTCGACACGC
TGCAGAACACGCAGCTAGATTAACCCTAGAAAGATAATCATATTGTGAC
GTACGTTAAAGATAATCATGTGTAAAATTGACGCATGTGTTTTATCGGT
CTGTATATCGAGGTTTATTTATTAATTTGAATAGATATTAAGTTTTATT
ATATTTACACTTACATACTAATAATAAATTCAACAAACAATTTATTTAT
GTTTATTTATTTATTAAAAAAAACAAAAACTCAAAATTTCTTCTATAAA
GTAACAAAACTTTTATGAGGGACAGCCCCCCCCCAAAGCCCCCAGGGAT
GTAATTACGTCCCTCCCCCGCTAGGGGGCAGCAGCGAGCCGCCCGGGGC
TCCGCTCCGGTCCGGCGCTCCCCCCGCATCCCCGAGCCGGCAGCGTGCG
GGGACAGCCCGGGCACGGGGAAGGTGGCACGGGATCGCTTTCCTCTGAA
CGCTTCTCGCTGCTCTTTGAGCCTGCAGACACCTGGGGGGATACGGGGA
AAAGGCCTCCACGGCC
3′TP
(SEQ ID NO: 3)
TTCCTGTCCTCACAGGAACGAAGTCCCTAAAGAAACAGTGGCAGCCAGG
TTTAGCCCCGGAATTGACTGGATTCCTTTTTTAGGGCCCATTGGTATGG
CTTTTTCCCCGTATCCCCCCAGGTGTCTGCAGGCTCAAAGAGCAGCGAG
AAGCGTTCAGAGGAAAGCGATCCCGTGCCACCTTCCCCGTGCCCGGGCT
GTCCCCGCACGCTGCCGGCTCGGGGATGCGGGGGGAGCGCCGGACCGGA
GCGGAGCCCCGGGCGGCTCGCTGCTGCCCCCTAGCGGGGGAGGGACGTA
ATTACATCCCTGGGGGCTTTGGGGGGGGGCTGTCCCTGATATCTATAAC
AAGAAAATATATATATAATAAGTTATCACGTAAGTAGAACATGAAATAA
CAATATAATTATCGTATGAGTTAAATCTTAAAAGTCACGTAAAAGATAA
TCATGCGTCATTTTGACTCACGCGGTCGTTATAGTTCAAAATCAGTGAC
ACTTACCGCATTGACAAGCACGCCTCACGGGAGCTCCAAGCGGCGACTG
AGATGTCCTAAATGCACAGCGACGGATTCGCGCTATTTAGAAAGAGAGA
GCAATATTTCAAGAATGCATGCGTCAATTTTACGCAGACTATCTTTCTA
GGGTTAATCTAGCTGCATCAGGATCATATCGTCGGGTCTTTTTTCCGGC
TCAGTCATCGCCCAAGCTGGCGCTATCTGGGCATCGGGGAGGAAGAAGC
CCGTGCCTTTTCCCGCGAGGTTGAAGCGGCATGGAAAGAGTTTGCCGAG
GATGACTGCTGCTGCATTGACGTTGAGCGAAAACGCACGTTTACCATGA
TGATTCGGGAAGGTGTGGCCATGCACGCCTTTAACGGTGAACTGTTCGT
TCAGGCCACCTGGGATACCAGTTCGTCGCGGCTTTTCCGGACACAGTTC
CGGATGGTCAGCCCGAAGCGCATCAGCAACCCGAACAATACCGGCGACA
GCCGGAACTGCCGTGCCGGTGTGCAGATTAATGACAGCGGTGCGGCGCT
GGGATATTACGTCAGCGAGGACGGGTATCCTGGCTGGATGCCGCAGAAA
TGGACATGGATA
ROIS
(SEQ ID NO: 5)
AATCTGAGCCAAGTAGAAGACCTTTTCCCCTCCTACCCCTACTTTCTAA
GTCACAGAGGCTTTTTGTTCCCCCAGACACTCTTGCAGATTAGTCCAGG
CAGAAACAGTTAGATGTCCCCAGTTAACCTCCTATTTGACACCACTGAT
TACCCCATTGATAGTCACACTTTGGGTTGTAAGTGACTTTTTATTTATT
TGTATTTTTGACTGCATTAAGAGGTCTCTAGTTTTTTACCTCTTGTTTC
CCAAAACCTAATAAGTAACTAATGCACAGAGCACATTGATTTGTATTTA
TTCTATTTTTAGACATAATTTATTAGCATGCATGAGCAAATTAAGAAAA
ACAACAACAAATGAATGCATATATATGTATATGTATGTGTGTACATATA
CACATATATATATATATTTTTTTTCTTTTCTTACCAGAAGGTTTTAATC
CAAATAAGGAGAAGATATGCTTAGAACTGAGGTAGAGTTTTCATCCATT
CTGTCCTGTAAGTATTTTGCATATTCTGGAGACGCAGGAAGAGATCCAT
CTACATATCCCAAAGCTGAATTATGGTAGACAAAGCTCTTCCACTTTTA
GTGCATCAATTTCTTATTTGTGTAATAAGAAAATTGGGAAAACGATCTT
CAATATGCTTACCAAGCTGTGATTCCAAATATTACGTAAATACACTTGC
AAAGGAGGATGTTTTTAGTAGCAATTTGTACTGATGGTATGGGGCCAAG
AGATATATCTTAGAGGGAGGGCTGAGGGTTTGAAGTCCAACTCCTAAGC
CAGTGCCAGAAGAGCCAAGGACAGGTACGGCTGTCATCACTTAGACCTC
ACCCTGTGGAGCCACACCCTAGGGTTGGCCAATCTACTCCCAGGAGCAG
GGAGGGCAGGAGCCAGGGCTGGGCATAAAAGTCAGGGCAGAGCCATCTA
TTGCTTACATTTGCTTCTGACACAACTGTGTTCACTAGCAACCTCAAAC
AGACACCATGGTGCACCTGACTCCTGAGGAGAAGTCTGCCGTTACTGCC
CTGTGGGGCAAGGTGAACGTG
ARS
(SEQ ID NO: 6)
TAGCTTGTATTTTTTGTAATTTAAAATAATGATGTATTAAAAACATTTG
TATTCTCTATATATATTTTAAATTTAGTTTAATTTCATAAACATTTCTC
AAGAGTATATTTTGTGCAGGGCATATTGCTAGTCATTATGGGATCTATA
TAGTTATGTTAAATTTAAAGTATGGTCTTACGGGGGAAGATGATAGAAA
ATGTACATTTATAAACTTCCTGCAATGTATGAGTTATTATGTTATAAAC
TTTTACATATTTTGACCCATTTAATCCCCATTTTGTAGATGAGTAGACT
GAGGCTCATGAAATGATAAAGATTTTCCCATGGTATCAGGAATAAGAGT
TGTCAAAGTAAAATTAAAACCAGGACTTTTGGCTCCCTAAAGCTATTCT
AATGCTATTATTTCAAGCATAAAGGCTAGTTTTTATGTAAGTTATAAAA
GAGATACACATTTAC
MIS
(SEQ ID NO: 7)
TACCACACAGTCTAAGCTGAACCTGGTTGGTTAACTTGAAAAATGCAGA
GATGTAGTTACATCAGCAGTGGGAAGACAAGAAGATCAGTTTCAGTGGG
AGAAGTCATTGCATTGGGAGGGGTAATTAACAGAGTGGTAGCATATGTG
GAATGTGGGCTCTATAGATAAGGACTGGCAGGAATGTTGTGTACCAGGG
CTGGGGGGATATAGAGGGTAAGGAAGTCTGGCCTTGAAATCAGGGAACA
AAGGACAACAAAACTTAAACGAGCTAAACCTTTGAAGAAGAATTTCTTA
CTGTAGTCAGCGATCATTATTGTAAACCTATGACAGTTCTTTCAAAATA
TTTTTCAGACTTGTCAACCGCTGTA

### (Example 2) Evaluation of Gene Expression Cassettes

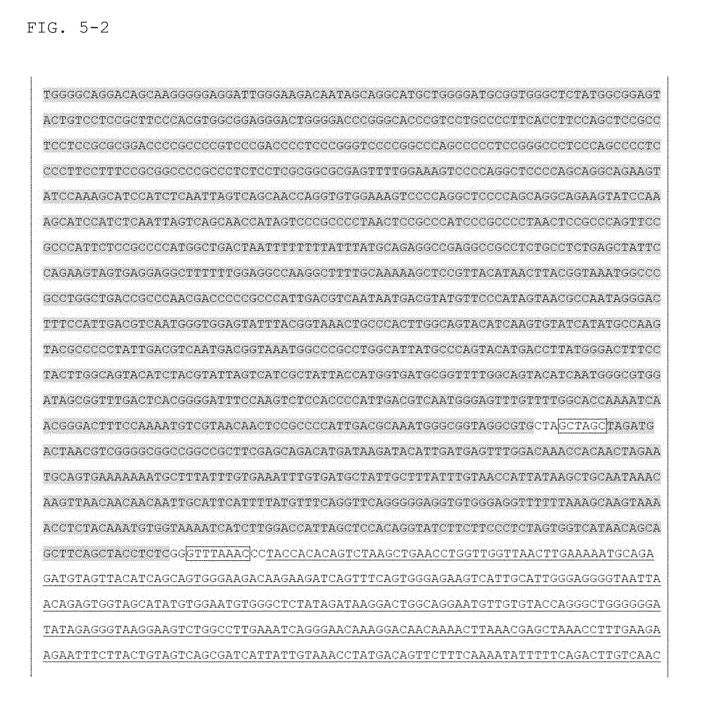

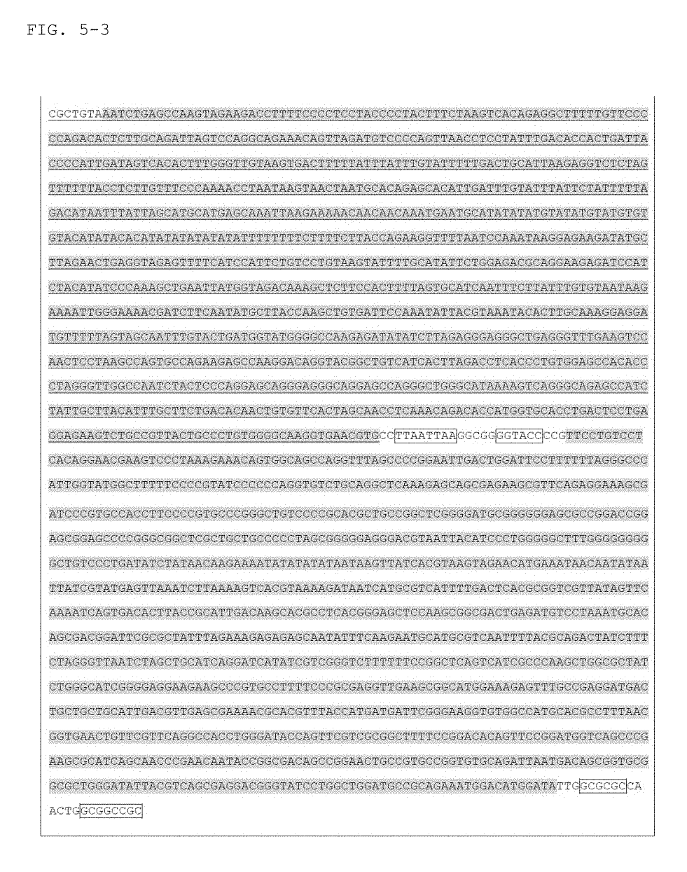

Expression vectors (gene expression vectors respectively corresponding to No. 1 to No. 10) containing the various gene expression cassettes No. 1 to No. 10 (FIG. 2) constructed in Example 1 were generated. A vector containing pCMViR-TSC illustrated in FIG. 2 was used as a control. The complete base sequence of the No. 4 gene expression vector, which is the highly efficient expression plasmid vector most effective in CHO cells out of No. 1 to No. 10, is set forth in FIG. 5 (SEQ ID NO: 8). In addition, the complete base sequence of the No. 1 gene expression vector, which is the highly efficient expression plasmid vector most effective in HEK293T cells, is set forth in FIG. 6 (SEQ ID NO: 9). In each gene expression cassette illustrated in FIG. 2, the sequence of cDNA encoding GFP-2A-Puro serving as the gene of interest was inserted in the forward direction using an EcoRI restriction enzyme site and an XbaI restriction enzyme site.

The gene expression vectors respectively corresponding to No. 1 to No. 10 and the control were each introduced into cells, and evaluated for the expression amount of the GFP fluorescent protein on the basis of a fluorescence intensity in accordance with the following procedure.

CHO cells (Chinese hamster ovary cells) and HEK293T cells (human embryonic kidney cells), which had been cultured in 10% FCS-containing GIBCO™ Dulbecco's Modified Eagle Medium/Nutrient Mixture F-12 (DMEM/F-12), were cultured in a 6 well plate until being 70% to 80% confluent. Through the use of FuGENE (trademark)-HD (gene transfection reagent), the gene expression vectors respectively corresponding to No. 1 to No. 10 and the control were each cotransfected with a transposase expression vector at 1:1 in terms of DNA amounts. The transposase expression vector carries a transposase gene in a pCMViR-TSC vector, which is a vector for transient expression, and when the vector is cotransfected with each of the gene expression vectors respectively corresponding to No. 1 to No. 10 carrying the transposon sequences (TP) of the present invention, the gene expression cassette is cleaved at the ends of the transposon sequences to efficiently insert the gene of interest at a TTAA site in a host genome. The cells transfected by such method were incubated for 24 hours, and then the GFP fluorescence intensities of the cells were measured using a fluorescence plate reader (FLUOROSKAN ASCENT FL, Thermo scientific) Cells having introduced therein no vector are represented by (−).

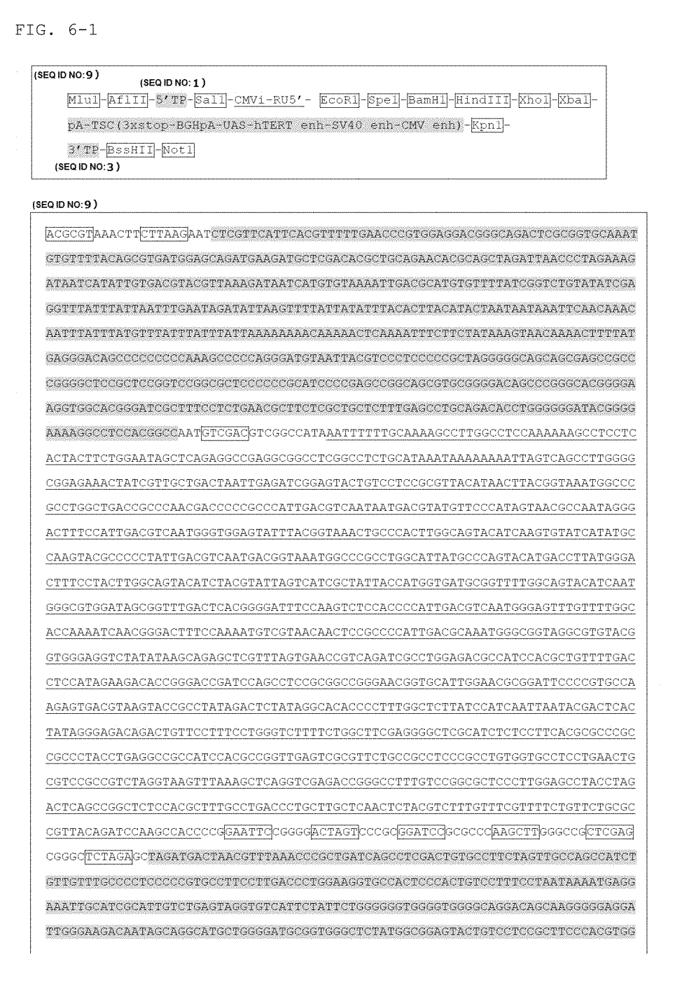

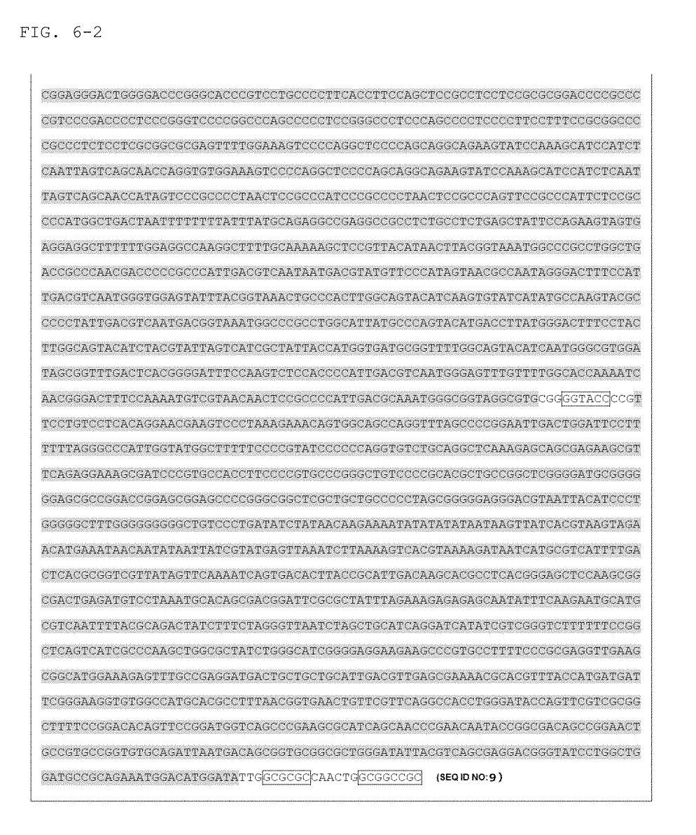

Further, CHO cells and HEK293T cells having similarly introduced therein the gene expression vectors respectively corresponding to No. 1 to No. 10 and the control were subjected to drug selection culture, starting after 48 hours of culture, with the addition of 10 μg/ml of puromycin (antibiotic) for 3 weeks, during which the medium was changed every 3 days. The fluorescence intensities of the cells after a lapse of 3 weeks of culture were measured with a fluorescence plate reader, and compared to the fluorescence intensities of the control and the cells cultured for 24 hours after transfection. The fluorescence intensities in the CHO cells are shown in FIG. 7, and the fluorescence intensities in the HEK293T cells are shown in FIG. 8. In the CHO cells, the GFP fluorescence intensity at 24 hours of culture of each of the control and the gene expression vectors respectively corresponding to No. 1 to No. 10 was found to hardly differ from that of (−), but after a lapse of 3 weeks, each of the cells having introduced therein the gene expression vectors respectively corresponding to No. 1 to No. 10 showed a clearly high GFP fluorescence intensity as compared to the control. In particular, the No. 4 gene expression vector showed a high value (FIG. 7). Meanwhile, in the HEK293T cells, the control was found to have transient expression by showing a high value for the GFP expression amount at 24 hours of culture, but each of the gene expression vectors respectively corresponding to No. 1 to No. 10 only showed a slightly higher value than (−). After a lapse of 3 weeks, in each of the cells having introduced therein the gene expression vectors respectively corresponding to No. 1 to No. 10, a tendency to show a high GFP fluorescence intensity was found as compared to the control, and in particular, the No. 1 gene expression vector showed a high value (FIG. 8).

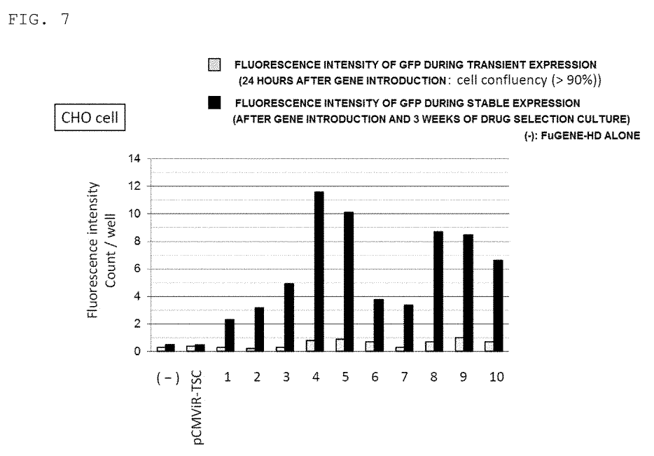

Next, the cells subjected to drug selection culture for 3 weeks were collected after fluorescence intensity measurement and subjected to protein quantification. On the basis of the resultant values, the fluorescence intensities of the cells were corrected, and the gene expression vectors respectively corresponding to No. 1 to No. 10 were compared and investigated on the basis of the corrected values. The results revealed that, in the CHO cells, the No. 4 gene expression vector showed a particularly high value (FIG. 9), and in the HEK293T cells, the No. 1 gene expression vector showed a particularly high value (FIG. 10).

The results of the foregoing confirmed that, in the CHO cells, the protein of interest was able to be stably produced over a long period of time through the use of the gene expression cassette of the present invention including the MIS sequence and the ROIS sequence or the ARS sequence upstream and downstream of the DNA construct (X) containing a gene of interest and a poly A addition sequence, and including the transposon sequences (T) further upstream and downstream. In addition, it was confirmed that, in the HEK293T cells, the protein of interest was able to be stably produced over a long period of time through the use of the gene expression cassette of the present invention including the transposon sequences (T) upstream and downstream of the DNA construct (X) containing a gene of interest and a poly A addition sequence.

### (Example 3) Production of Human Histidine-Rich Glycoprotein (HRG)

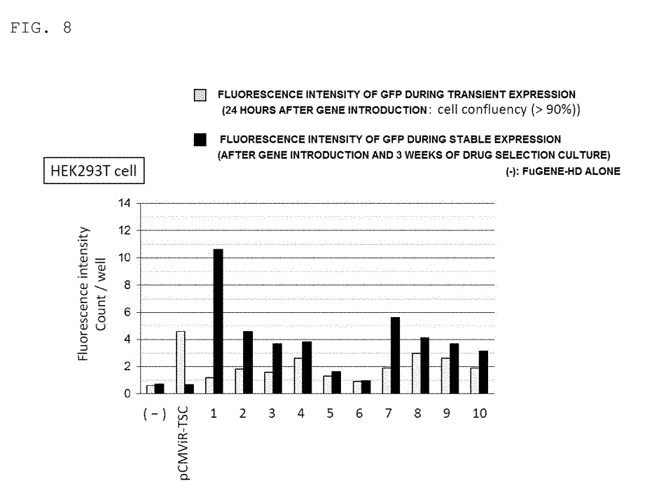

In this Example, recombinant human HRG was generated as described below. A No. 4-HRG expression vector was generated using the gene expression cassette No. 4, which was the highly efficient gene expression cassette most effective in the CHO cells described in Example 1, and using, as the gene of interest, DNA encoding the coding region of human HRG set forth in SEQ ID NO: 10 (DNA formed of a base sequence identified by GenBank Accession No. BC069574 (NCBI) (see FIG. 11). Specifically, CHO cells (Chinese hamster ovary cells), which had been cultured in 10% FCS-containing GIBCO™ Dulbecco's Modified Eagle Medium/Nutrient Mixture F-12 (DMEM/F-12), were cotransfected with the No. 4-HRG expression vector, a transposase expression vector, and the No. 4 gene expression vector described in Example 1, which served as a drug resistance gene expression vector, at 5:4:1 in terms of DNA amounts through the use of FuGENE (trademark)-HD (gene transfection reagent). Starting after 48 hours of culture following the gene introduction, drug selection culture was performed with the addition of 10 μg/ml of puromycin (antibiotic) for 3 weeks, during which the medium was changed every 3 days.

Amino Acid Sequence of Mature HRG
(SEQ ID NO: 10)
VSPTDCSAVEPEAEKALDLINKRRRDGYLFQLLRIADAHLDRVENTTVY
YLVLDVQESDCSVLSRKYWNDCEPPDSRRPSEIVIGQCKVIATRHSHES
QDLRVIDFNCTTSSVSSALANTKDSPVLIDFFEDTERYRKQANKALEKY
KEENDDFASFRVDRIERVARVRGGEGTGYFVDFSVRNCPRHHFPRHPNV
FGFCRADLFYDVEALDLESPKNLVINCEVFDPQEHENINGVPPHLGHPF
HWGGHERSSTTKPPFKPHGSRDHHHPHKPHEHGPPPPPDERDHSHGPPL
PQGPPPLLPMSCSSCQHATFGTNGAQRHSHNNNSSDLHPHKHHSHEQHP
HGHHPHAHHPHEHDTHRQHPHGHHPHGHHPHGHHPHGHHPHGHHPHCHD
FQDYGPCDPPPHNQGHCCHGHGPPPGHLRRRGPGKGPRPFHCRQIGSVY
RLPPLRKGEVLPLPEANFPSFPLPHHKHPLKPDNQPFPQSVSESCPGKF
KSGFPQVSMFFTHTFPK

A culture supernatant containing recombinant human HRG was collected. A QIAGEN™ Ni-NTA agarose gel (gel obtained by binding Ni-NTA to a Sepharose CL-6B support), which had been washed in advance with 30 ml of PBS (−), was added to the culture supernatant, and rotating incubation was performed at 4° C. for 2 hours to bind the recombinant human HRG to the QIAGEN™ Ni-NTA agarose gel. The QIAGEN™ Ni-NTA agarose gel was transferred to a purification column, and then the column was sequentially washed with a washing liquid 1 (PBS(−) containing 30 mM imidazole (pH 7.4)), a washing liquid 2 (1 M NaCl+10 mM PB (pH 7.4)), and a washing liquid 3 (PBS(−) (pH 7.4)). The recombinant human HRG was eluted with PBS(−) containing 500 mM imidazole (pH 7.4) at 4° C. The purified product was confirmed to be HRG by protein staining after western blot and SDS-PAGE.

For each of the HRG generated by the gene recombination technique of the present invention described above (hereinafter referred to as “recombinant HRG”) and human plasma-derived HRG generated in Example 1 of WO 2013/183494 A1 (hereinafter referred to as “plasma-derived HRG”), activity of inducing a spherical morphology of neutrophils (sphere-forming activity), and effects on the survival rate of CLP sepsis model mice and production-suppressing activity on a reactive oxygen molecular species were confirmed.

### (Experimental Example 3-1) Morphology of Neutrophils

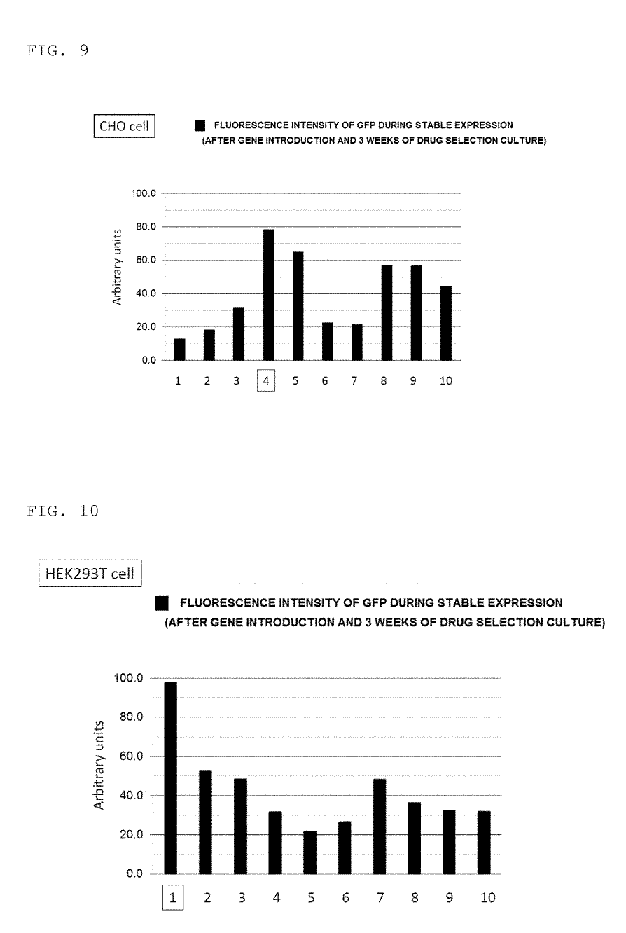

In accordance with a flow chart illustrated in FIG. 5 of WO 2013/183494 A1, the morphology of neutrophils in a system obtained by adding 50 μl of HRG: 2 μM, final concentration: 1 μM) to 50 μl of a neutrophil suspension (5×10⁵ cells/ml) was observed with a fluorescence microscope through fluorescence labeling of the cells with Calcein, and the sphere-forming activity of each HRG was confirmed on the basis of the sphere-forming rate (%) of the neutrophils. The results confirmed that each of the recombinant HRG and the plasma-derived HRG had similar sphere-forming activity (FIG. 12).

### (Experimental Example 3-2) Effect of HRG on CLP Sepsis Model Mice

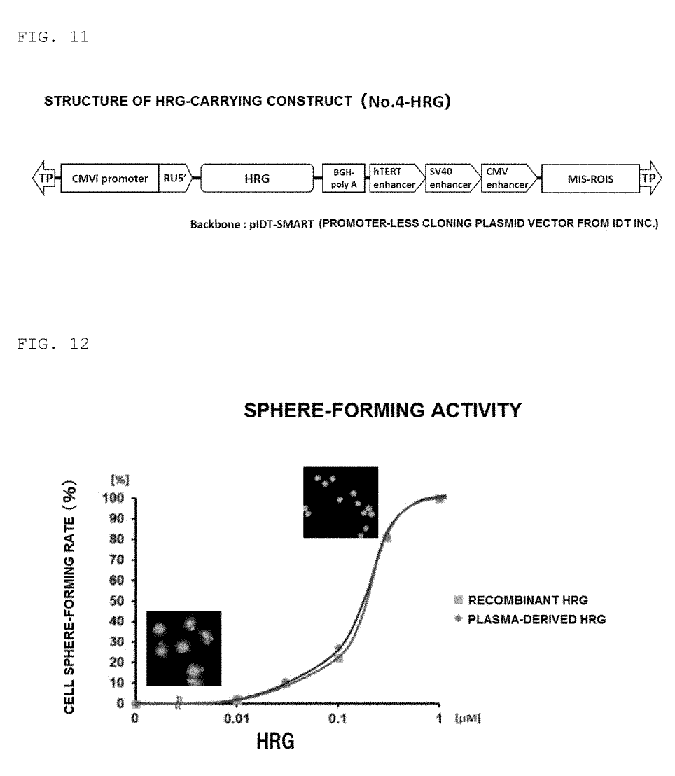

In this Experimental Example, a survival rate based on a Kaplan-Meier method was investigated with a sepsis model with cecal ligation and puncture (CLP). A cecum was excised from the abdominal cavity of a mouse, the root of the cecum was ligated with a suture, and the layer of the cecal wall was punctured using an 18-gauge syringe needle to produce a CLP sepsis model. A sham mouse was used as a control. At 5 minutes, 24 hours, and 48 hours after the surgery, the recombinant HRG (HRG: 400 μg/mouse) was injected into the tail vein (n=10). HSA and PBS(−) were used as controls (n=10). As a result, the results of analysis by the Kaplan-Meier method revealed that the recombinant HRG-administered group was confirmed to have a significantly high cumulative survival rate (FIG. 13).

### (Experimental Example 3-3) Production-Suppressing Activity on Reactive Oxygen Molecular Species

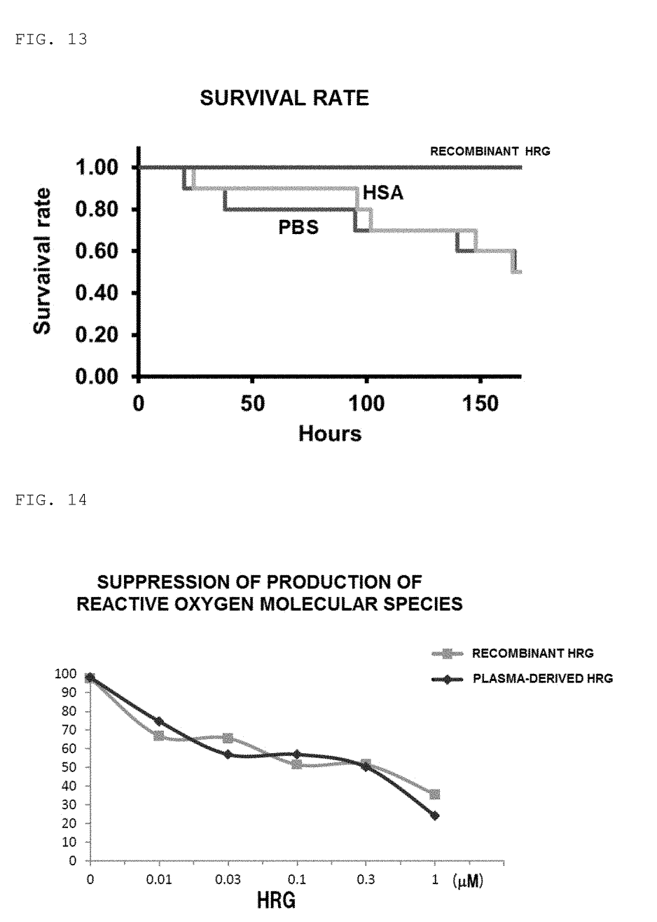

Isolated human neutrophils were incubated with the addition of isoluminol (final concentration: 50 mM) and horse radish peroxidase type IV (final concentration: 4 U/ml), and the level of a reactive oxygen molecular species released to the outside of the cells was measured on the basis of chemiluminescence 15 minutes after reaction initiation. The level in the absence of HRG was defined as 100%, and values in the presence of HRG at concentrations of from 0.01 μM to 1.0 μM were calculated to be expressed in % (FIG. 14). The results revealed that the recombinant HRG showed production-suppressing activity on the reactive oxygen molecular species approximately equal to that of the HRG purified from human plasma.

### (Example 4) Production of Recombinant PD-1

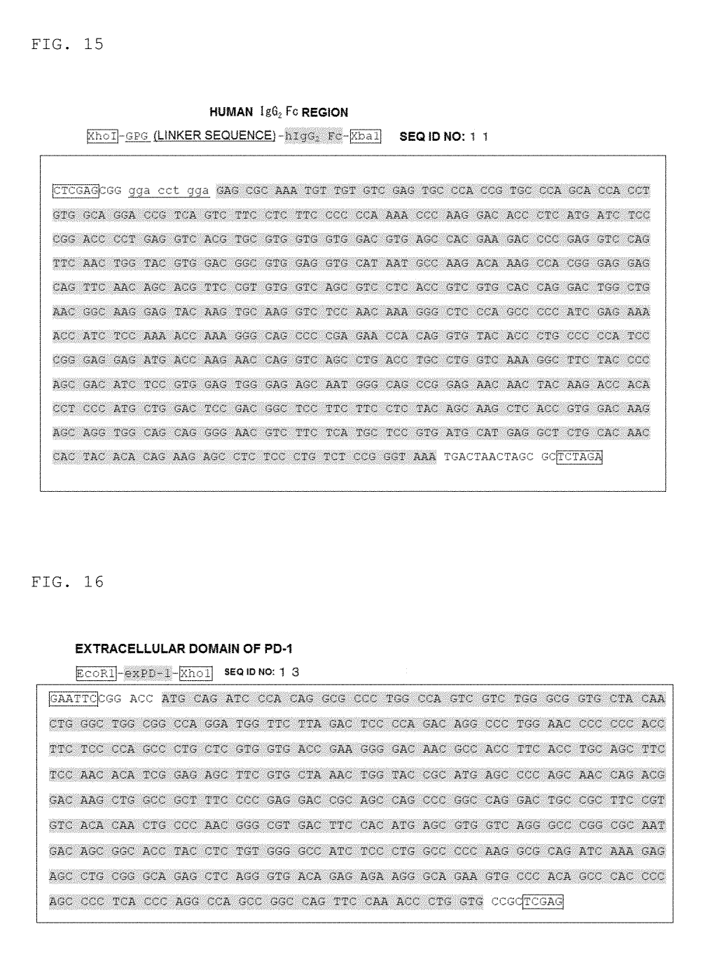

In this Example, the production of a PD-1 extracellular domain by a gene recombination operation is described. In this Example, a gene expression vector obtained by linking a TP sequence upstream of a promoter, a DNA construct containing a gene to be expressed and a poly A addition sequence downstream of the promoter, an enhancer downstream of the DNA construct, and an MIS sequence, an ROIS sequence, and a TP sequence downstream of the enhancer was used to generate CHO cells that stably and highly produced the protein of interest, and the cells were used to produce a protein serving as a pharmaceutical product candidate with high efficiency. The structure of the construct used is illustrated in FIG. 1(B). The construct No. 4 of FIG. 2 was used to insert the gene to be expressed as an inserted gene. Each of all proteins serving as pharmaceutical product candidates is fused with an Fc region of human IgG, which is part of an antibody, and hence is considered to be a pharmaceutical product that: improves the stability of a protein preparation that is generally considered to have low in vivo stability and to hardly provide drug efficacy; and by virtue of the use of the Fc region of human IgG₂, has low complement activity, resulting in alleviation of an inflammatory response that is a side effect. The complete base sequence of the human IgG₂ Fc region is illustrated in FIG. 15, and a base sequence containing a linker and a restriction enzyme recognition site is set forth in SEQ ID NO: 11 of the sequence listing. The amino acid sequence of the human IgG₂ Fc region is set forth in SEQ ID NO: 12 of the sequence listing.

Amino Acid Sequence of Human IgG₂ Fc Region
(SEQ ID NO: 12)
ERKCCVECPPCPAPPVAGPSVFLFPPKPKDTLMISRTPEVTCVVVDVSH
EDPEVQFNWYVDGVEVHNAKTKPREEQFNSTFRVVSVLTVVHQDWLNGK
EYKCKVSNKGLPAPIEKTISKTKGQPREPQVYTLPPSREEMTKNQVSLT
CLVKGFYPSDISVEWESNGQPENNYKTTPPMLDSDGSFFLYSKLTVDKS
RWQQGNVFSCSVMHEALHNHYTQKSLSLSPG

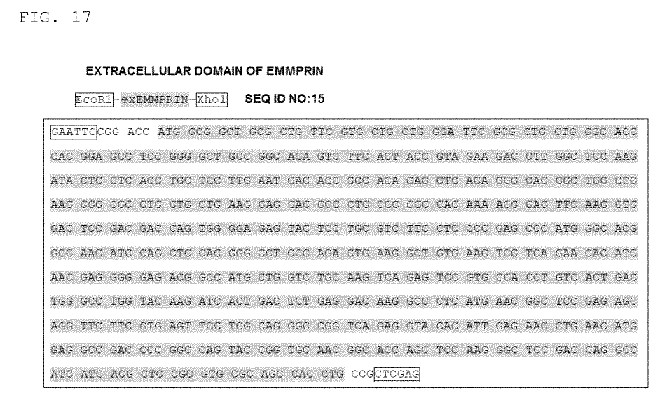

The extracellular domain of recombinant PD-1 was generated as described below. Through the use of the gene expression cassette No. 4, which was the highly efficient gene expression cassette most effective in CHO cells described in Example 1, the base sequence of DNA encoding the coding region of the PD-1 extracellular domain illustrated in FIG. 16 (DNA formed of a base sequence identified by GenBank Accession No. BC074740 (NCBI)) was fused with a sequence encoding the Fc region of human IgG₂ illustrated in FIG. 15 to generate a polynucleotide (exPD-1-Fc) as the gene of interest. A No. 4-exPD-1-Fc expression vector having PD-1-Fc in place of the HRG of the HRG-carrying construct illustrated in FIG. 11 was generated. exPD-1-Fc was transfected by the same technique as in Example 3.

Fc fusion protein-highly producing CHO cells in the logarithmic growth phase were prepared at a concentration of 5×10⁵ cells/ml, seeded at 500 ml into HYPERFlask (Corning), and cultured at 37° C. in the presence of 5% CO₂ for 10 days through the use of CD-CHO Medium (Life Technologies), and the culture supernatant was collected. The culture supernatant was added to a column packed with Protein G Sepharose 4 Fast Flow (GE Healthcare), which had been washed in advance with 20 mM sodium phosphate (pH 7.0), to bind the Fc fusion protein. A non-specifically bound protein was washed out with 20 mM sodium phosphate (pH 7.0). The Fc fusion protein was eluted with 0.1 M Glycine-HCl (pH 2.7), and the eluate was neutralized with 1 M Tris-HCl (pH 9.0). The Fc fusion protein generated in this Example is referred to as exPD-1-Fc. The amino acid sequence of the mature PD-1 extracellular domain is set forth in SEQ ID NO: 14 of the sequence listing.

Amino Acid Sequence of Mature PD-1 Extracellular
Domain
(SEQ ID NO: 14)
GWFLDSPDRPWNPPTFSPALLVVTEGDNATFTCSFSNTSESFVLNWYRM
SPSNQTDKLAAFPEDRSQPGQDCRFRVTQLPNGRDFHMSVVRARRNDSG
TYLCGAISLAPKAQIKESLRAELRVTERRAEVPTAHPSPSPRPAGQFQT
LV

### (Example 5) Production of Recombinant EMMPRIN

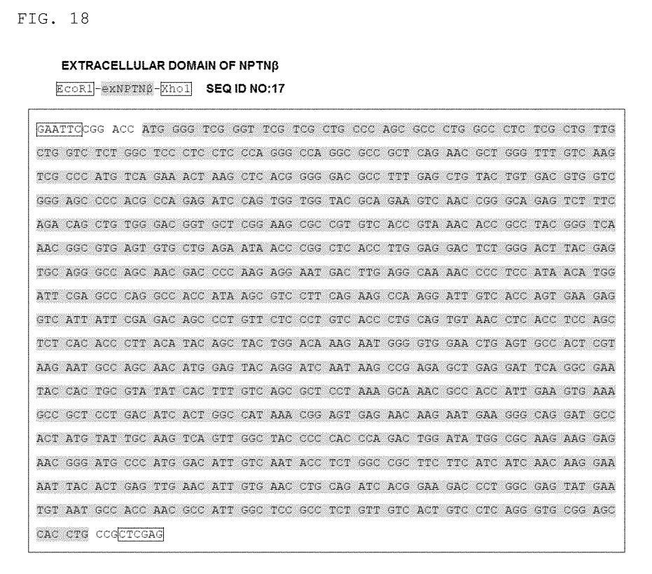

In this Example, the production of an EMMPRIN extracellular domain by a gene recombination operation is described. An Fc fusion protein of EMMPRIN extracellular domain+human IgG₂ was generated by performing a gene recombination operation by the same technique as in Example 4 except that a polynucleotide (exEMMPRIN-Fc) was generated by fusing the base sequence of DNA encoding the coding region of the EMMPRIN extracellular domain illustrated in FIG. 17 (DNA formed of a base sequence identified by GenBank Accession No. ENSG00000172270 (Ensembl)) with a sequence encoding the Fc region of human IgG₂ illustrated in FIG. 15, and was used as the gene of interest. The Fc fusion protein generated in this Example is referred to as exEmmprin-Fc. The amino acid sequence of the EMMPRIN extracellular domain is set forth in SEQ ID NO: 16 of the sequence listing.

Amino Acid Sequence of Mature EMMPRIN
Extracellular Domain
(SEQ ID NO: 16)
ASGAAGTVFTTVEDLGSKILLTCSLNDSATEVTGHRWLKGGVVLKEDAL
PGQKTEFKVDSDDQWGEYSCVFLPEPMGTANIQLHGPPRVKAVKSSEHI
NEGETAMLVCKSESVPPVTDWAWYKITDSEDKALMNGSESRFFVSSSQG
RSELHIENLNMEADPGQYRCNGTSSKGSDQAIITLRVRSHL

### (Example 6) Production of Recombinant NPTNβ

In this Example, the production of an NPTNβ extracellular domain by a gene recombination operation is described.

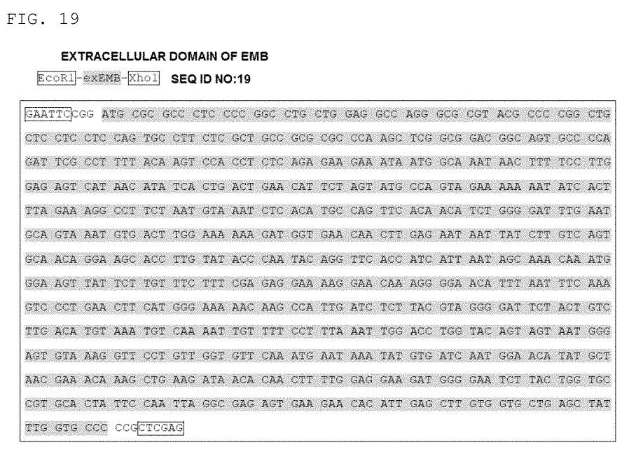

In this Example, an Fc fusion protein of NPTNβ extracellular domain+human IgG₂ was generated by performing a gene recombination operation by the same technique as in Example 4 except that a polynucleotide (exNPTNβ-Fc) was generated by fusing the base sequence of DNA encoding the coding region of the NPTNβ extracellular domain illustrated in FIG. 18 (DNA formed of a base sequence identified by GenBank Accession No. ENSG00000156642 (Ensembl)) with a sequence encoding the Fc region of human IgG₂ illustrated in FIG. 15, and was used as the gene of interest. The Fc fusion protein generated in this Example is referred to as exENPTNβ-Fc. The amino acid sequence of the NPTNβ extracellular domain is set forth in SEQ ID NO: 18 of the sequence listing.

Amino Acid Sequence of Mature NPTNβ Extracellular
Domain
(SEQ ID NO: 18)
QNAGFVKSPMSETKLTGDAFELYCDVVGSPTPEIQWWYAEVNRAESFRQ
LWDGARKRRVTVNTAYGSNGVSVLRITRLTLEDSGTYECRASNDPKRND
LRQNPSITWIRAQATISVLQKPRIVTSEEVIIRDSPVLPVTLQCNLTSS
SHTLTYSYWTKNGVELSATRKNASNMEYRINKPRAEDSGEYHCVYHFVS
APKANATIEVKAAPDITGHKRSENKNEGQDATMYCKSVGYPHPDWIWRK
KENGMPMDIVNTSGRFFIINKENYTELNIVNLQITEDPGEYECNATNAI
GSASVVTVLRVRSHL

### (Example 7) Production of Recombinant EMB

In this Example, the production of an EMB extracellular domain by a gene recombination operation is described. An Fc fusion protein of EMB extracellular domain+human IgG₂ was generated by performing a gene recombination operation by the same technique as in Example 4 except that a polynucleotide (exEMBβ-Fc) was generated by fusing the base sequence of DNA encoding the coding region of the EMB extracellular domain illustrated in FIG. 19 (DNA formed of a base sequence identified by GenBank Accession No. BC059398 (NCBI)) with a sequence encoding the Fc region of human IgG₂ illustrated in FIG. 15, and was used as the gene of interest. The Fc fusion protein generated in this Example is referred to as exEMB-Fc. The amino acid sequence of the EMB extracellular domain is set forth in SEQ ID NO: 20 of the sequence listing.

Amino Acid Sequence of Mature EMB Extracellular
Domain
(SEQ ID NO: 20)
DGSAPDSPFTSPPLREEIMANNFSLESHNISLTEHSSMPVEKNITLERP
SNVNLTCQFTTSGDLNAVNVTWKKDGEQLENNYLVSATGSTLYTQYRFT
IINSKQMGSYSOFFREEKEQRGTFNFKVPELHGKNKPLISYVGDSTVLT
CKCQNCFPLNWTWYSSNGSVKVPVGVQMNKYVINGTYANETKLKITQLL
EEDGESYWCRALFQLGESEEHIELVVLSYLVP

### (Example 8) Production of Recombinant RAGE

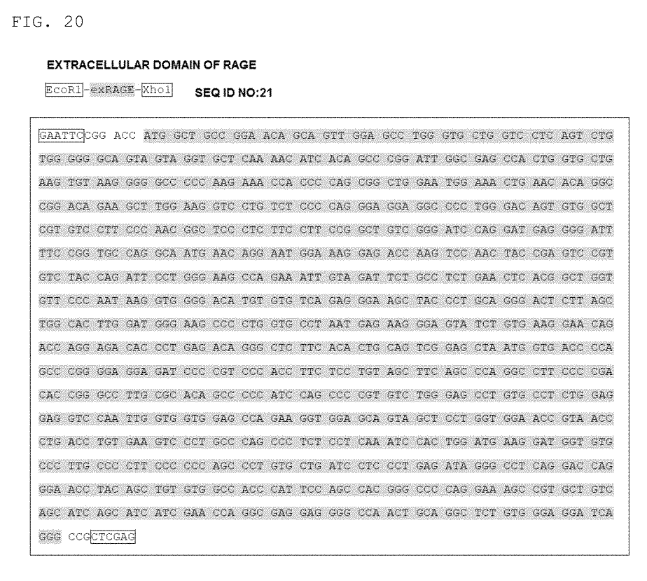

In this Example, the production of an RAGE extracellular domain by a gene recombination operation is described. An Fc fusion protein of RAGE extracellular domain+human IgG₂ was generated by performing a gene recombination operation by the same technique as in Example 4 except that a polynucleotide (exRAGE-Fc) was generated by fusing the base sequence of DNA encoding the coding region of the RAGE extracellular domain illustrated in FIG. 20 (DNA formed of a base sequence identified by GenBank Accession No. ENSG00000204305 (Ensembl)) with a sequence encoding the Fc region of human IgG₂ illustrated in FIG. 15, and was used as the gene of interest. The Fc fusion protein generated in this Example is referred to as exRAGE-Fc. The amino acid sequence of the RAGE extracellular domain is set forth in SEQ ID NO: 22 of the sequence listing.

Amino Acid Sequence of Mature RAGE Extracellular
Domain
(SEQ ID NO: 22)
QNITARIGEPLVLKCKGAPKKPPQRLEWKLNTGRTEAWKVLSPQGGGPW
DSVARVLPNGSLFLPAVGIQDEGIFRCQAMNRNGKETKSNYRVRVYQIP
GKPEIVDSASELTAGVPNKVGTCVSEGSYPAGTLSWHLDGKPLVPNEKG
VSVKEQTRRHPETGLFTLQSELMVTPARGGDPRPTFSCSFSPGLPRHRA
LRTAPIQPRVWEPVPLEEVQLVVEPEGGAVAPGGTVTLTCEVPAQPSPQ
IHWMKDGVPLPLPPSPVLILPEIGPQDQGTYSCVATHSSHGPQESRAVS
ISIIEPGEEGPTAGSVGGSGLGTLA

### (Example 9) Production of Recombinant MCAM

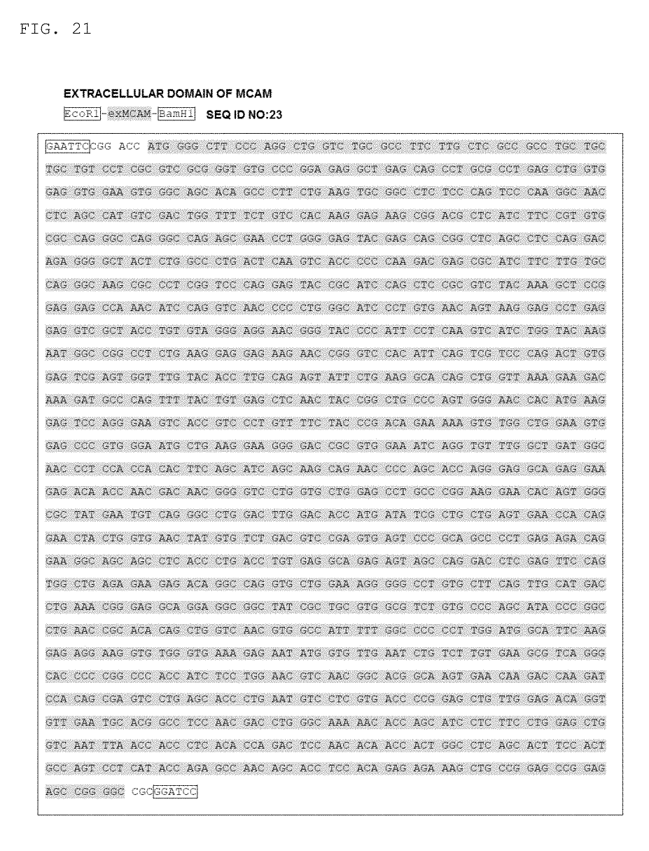

In this Example, the production of an MCAM extracellular domain by a gene recombination operation is described. An Fc fusion protein of MCAM extracellular domain+human IgG₂ was generated by performing a gene recombination operation by the same technique as in Example 4 except that a polynucleotide (exMCAM-Fc) was generated by fusing the base sequence of DNA encoding the coding region of the MCAM extracellular domain illustrated in FIG. 21 (DNA formed of a base sequence identified by GenBank Accession No. BC056418 (NCBI)) with a sequence encoding the Fc region of human IgG₂ illustrated in FIG. 15, and was used as the gene of interest. The Fc fusion protein generated in this Example is referred to as exMCAM-Fc. The amino acid sequence of the MCAM extracellular domain is set forth in SEQ ID NO: 24 of the sequence listing.

Amino Acid Sequence of Mature MCAM Extracellular
Domain
(SEQ ID NO: 24)
VPGEAEQPAPELVEVEVGSTALLKCGLSQSQGNLSHVDWFSVHKEKRTL
IFRVRQGQGQSEPGEYEQRLSLQDRGATLALTQVTPQDERIFLCQGKRP
RSQEYRIQLRVYKAPEEPNIQVNPLGIPVNSKEPEEVATCVGRNGYPIP
QVIWYKNGRPLKEEKNRVHIQSSQTVESSGLYTLQSILKAQLVKEDKDA
QFYCELNYRLPSGNHMKESREVTVPVFYPTEKVWLEVEPVGMLKEGDRV
EIRCLADGNPPPHFSISKQNPSTREAEEETTNDNGVLVLEPARKEHSGR
YECQGLDLDTMISLLSEPQELLVNYVSDVRVSPAAPERQEGSSLTLTCE
AESSQDLEFQWLREETGQVLERGPVLQLHDLKREAGGGYRCVASVPSIP
GLNRTQLVNVAIFGPPWMAFKERKVWVKENMVLNLSCEASGHPRPTISW
NVNGTASEQDQDPQRVLSTLNVLVTPELLETGVECTASNDLGKNTSILF
LELVNLTTLTPDSNTTTGLSTSTASPHTRANSTSTERKLPEPESRG

### (Example 10) Production of Recombinant ALCAM

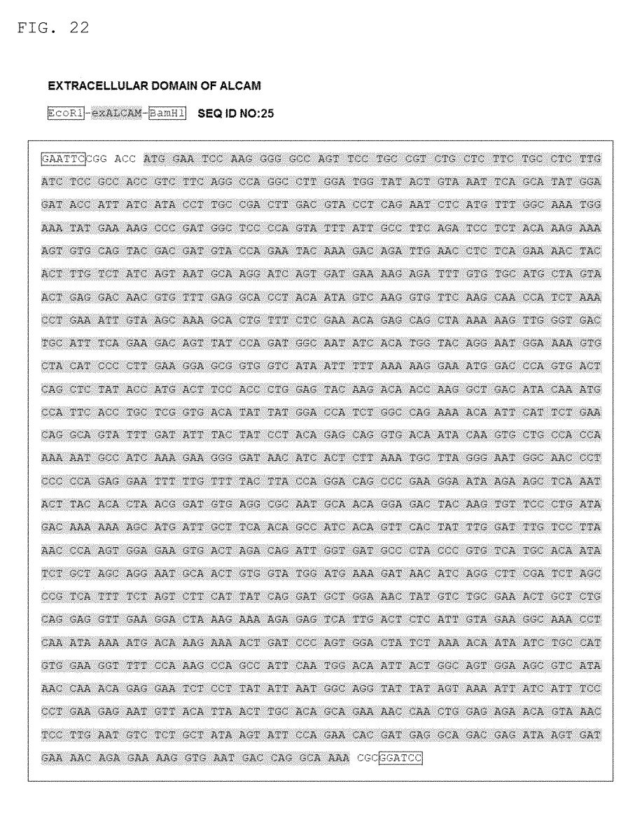

In this Example, the production of an ALCAM extracellular domain by a gene recombination operation is described. An Fc fusion protein of ALCAM extracellular domain+human IgG₂ was generated by performing a gene recombination operation by the same technique as in Example 4 except that a polynucleotide (exALCAM-Fc) was generated by fusing the base sequence of DNA encoding the coding region of the ALCAM extracellular domain illustrated in FIG. 22 (DNA formed of a base sequence identified by GenBank Accession No. BC137097 (NCBI)) with a sequence encoding the Fc region of human IgG₂ illustrated in FIG. 15, and was used as the gene of interest. The Fc fusion protein generated in this Example is referred to as exALCAM-Fc. The amino acid sequence of the ALCAM extracellular domain is set forth in SEQ ID NO: 26 of the sequence listing.

Amino Acid Sequence of Mature ALCAM Extracellular
Domain
(SEQ ID NO: 26)
WYTVNSAYGDTIIIPCRLDVPQNLMFGKWKYEKPDGSPVFIAFRSSTKK
SVQYDDVPEYKDRLNLSENYTLSISNARISDEKRFVCMLVTEDNVFEAP
TIVKVFKQPSKPEIVSKALFLETEQLKKLGDCISEDSYPDGNITWYRNG
KVLHPLEGAVVIIFKKEMDPVTQLYTMTSTLEYKTTKADIQMPFTCSVT
YYGPSGQKTIHSEQAVFDIYYPTEQVTIQVLPPKNAIKEGDNITLKCLG
NGNPPPEEFLFYLPGQPEGIRSSNTYTLTDVRRNATGDYKCSLIDKKSM
IASTAITVHYLDLSLNPSGEVTRQIGDALPVSCTISASRNATVVWMKDN
IRLRSSPSFSSLHYQDAGNYVCETALQEVEGLKKRESLTLIVEGKPQIK
MTKKTDPSGLSKTIICHVEGFPKPAIQWTITGSGSVINQTEESPYINGR
YYSKIIISPEENVTLTCTAENQLERTVNSLNVSAISIPEHDEADEISDE
NREKVNDQAK

### (Example 11) Production of Recombinant ErbB2

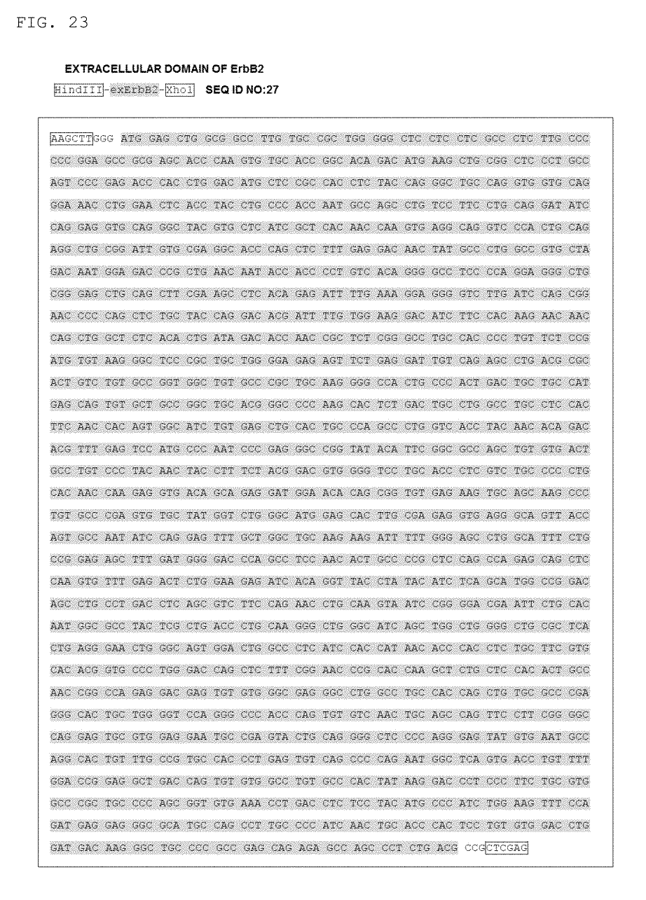

In this Example, the production of an ErbB2 extracellular domain by a gene recombination operation is described. An Fc fusion protein of ErbB2 extracellular domain+human IgG₂ was generated by performing a gene recombination operation by the same technique as in Example 4 except that a polynucleotide (exErbB2-Fc) was generated by fusing the base sequence of DNA encoding the coding region of the ErbB2 extracellular domain illustrated in FIG. 23 (DNA formed of a base sequence identified by GenBank Accession No. ENSG00000141736 (Ensembl)) with a sequence encoding the Fc region of human IgG₂ illustrated in FIG. 15, and was used as the gene of interest. The Fc fusion protein generated in this Example is referred to as exErbB2-Fc. The amino acid sequence of the ErbB2 extracellular domain is set forth in SEQ ID NO: 28 of the sequence listing.

Amino Acid Sequence of Mature ErbB2 Extracellular
Domain
(SEQ ID NO: 28)
TQVCTGTDMKLRLPASPETHLDMLRHLYQGCQVVQGNLELTYLPTNASL
SFLQDIQEVQGYVLIAHNQVRQVPLQRLRIVRGTQLFEDNYALAVLDNG
DPLNNTTPVTGASPGGLRELQLRSLTEILKGGVLIQRNPQLCYQDTILW
KDIFHKNNQLALTLIDTNRSRACHPCSPMCKGSRCWGESSEDCQSLTRT
VCAGGCARCKGPLPTDCCHEQCAAGCTGPKHSDCLACLHFNHSGICELH
CPALVTYNTDTFESMPNPEGRYTFGASCVTACPYNYLSTDVGSCTLVCP
LHNQEVTAEDGTQRCEKCSKPCARVCYGLGMEHLREVRAVTSANIQEFA
GCKKIFGSLAFLPESFDGDPASNTAPLQPEQLQVFETLEEITGYLYISA
WPDSLPDLSVFQNLQVIRGRILHNGAYSLTLQGLGISWLGLRSLRELGS
GLALIHHNTHLCFVHTVPWDQLFRNPHQALLHTANRPEDECVGEGLACH
QLCARGHCWGPGPTQCVNCSQFLRGQECVEECRVLQGLPREYVNARHCL
PCHPECQPQNGSVTCFGPEADQCVACAHYKDPPFCVARCPSGVKPDLSY
MPIWKFPDEEGACQPCPINCTHSCVDLDDKGCPAEQRASPLT

### (Example 12) Production of Recombinant HRG

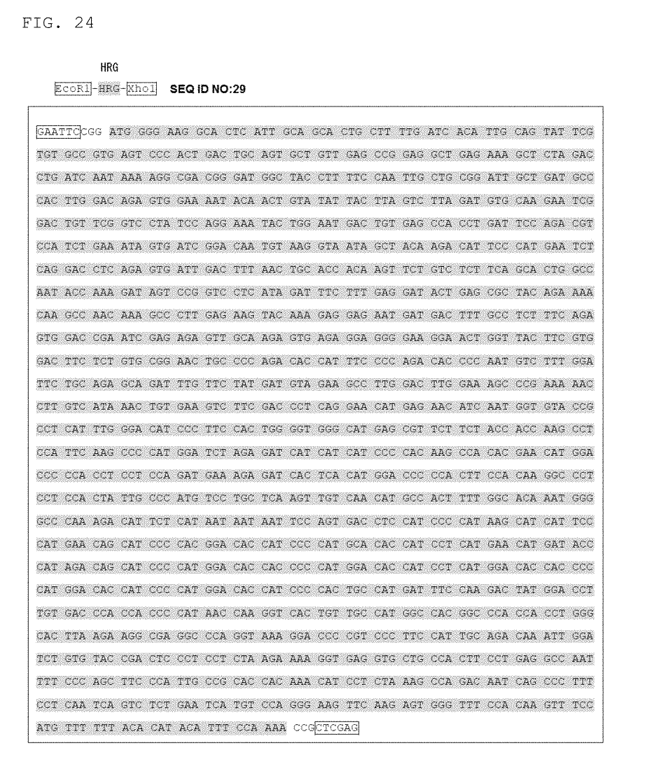

In this Example, an Fc fusion protein of HRG+human IgG₂ was generated by performing a gene recombination operation by the same technique as in Example 4 except that a polynucleotide (HRG-Fc) was generated by fusing the base sequence of DNA encoding the coding region of the HRG illustrated in FIG. 24 (DNA formed of abase sequence identified by GenBank Accession No. BC069574 (NCBI)) with a sequence encoding the Fc region of human IgG₂ illustrated in FIG. 15, and was used as the gene of interest. The Fc fusion protein generated in this Example is referred to as HRG-Fc. The amino acid sequence of the HRG is set forth in SEQ ID NO: 10 of the sequence listing in the same manner as in Example 3.

### (Experimental Example 4) Protein Expression Amount

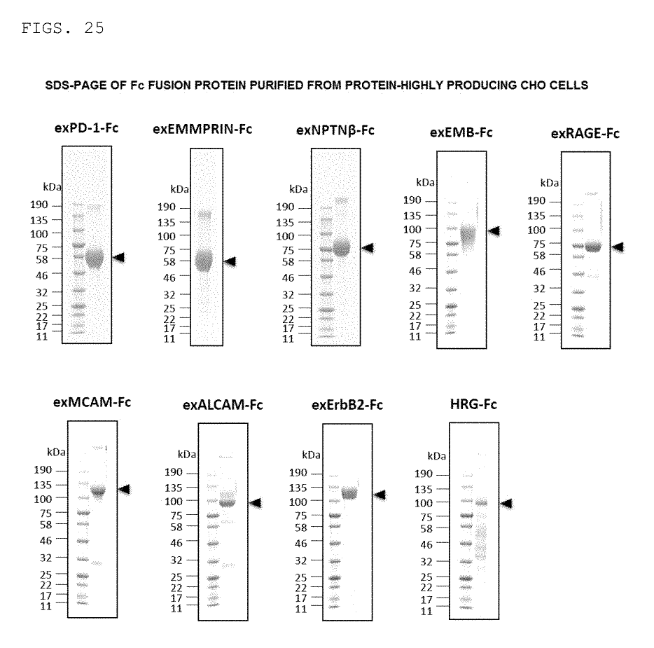

The purified Fc fusion proteins generated by the methods of Examples 4 to 12 were separated using SDS-PAGE, and the purity of each of the Fc fusion proteins was confirmed by CBB staining (FIG. 25). Further, the protein amount of each of the purified Fc fusion proteins was quantified by a Bradford method, and a purified protein amount in 500 ml culture was calculated and shown in Table 1.

TABLE 1
Protein amount after purification of 500 mLmp
culture supernatant with Protein G sepharose

| Fc fusion protein | Protein amount |
|---|---|
| exPD-1-Fc | 11.6 mg |
| exEMMPRIN-Fc | 24.2 mg |
| exNPTNβ-Fc | 75.3 mg |
| exEMB-Fc | 2.4 mg |
| exRAGE-Fc | 9.4 mg |
| exMCAM-Fc | 19.6 mg |
| exALCAM-Fc | 4.3 mg |
| exErbB2-Fc | 2.3 mg |
| HRG-Fc | 9.7 mg |

### (Experimental Example 5) Sphere-Forming Activity of HRG on Neutrophils

In this Experimental Example, an ability to induce a spherical morphology of neutrophils (sphere-forming activity) was confirmed as HRG activity. For each of the recombinant human HRG (rHRG) generated in Example 3 herein, the HRG-Fc generated in Example 12 herein, and the plasma-derived HRG (hHRG) generated in Example 3 herein, the HRG activity was measured, and their respective activities were compared.

Neutrophils were isolated from blood collected from the human cubital vein (anticoagulant: heparin) using a separation blood cell-separating solution Polymorphprep™ (Cosmo Bio Co., Ltd.) by a conventional method, and were adjusted with PBS(−) to 2×10⁶/ml. To 3 ml of the cell suspension, 1.5 μl of a Hoechst dye (10 mg/ml) serving as a nuclear staining dye and 3 μl of Calcein-AM (5 mM) serving as a fluorescent staining dye suited for staining living cells were added, followed by incubation at 37° C. for 15 minutes. After centrifugation, the supernatant was removed, followed by suspension in 6 ml of Hank's Balanced Salt Solution (HBSS) to prepare a 1×10⁶/ml neutrophil suspension.

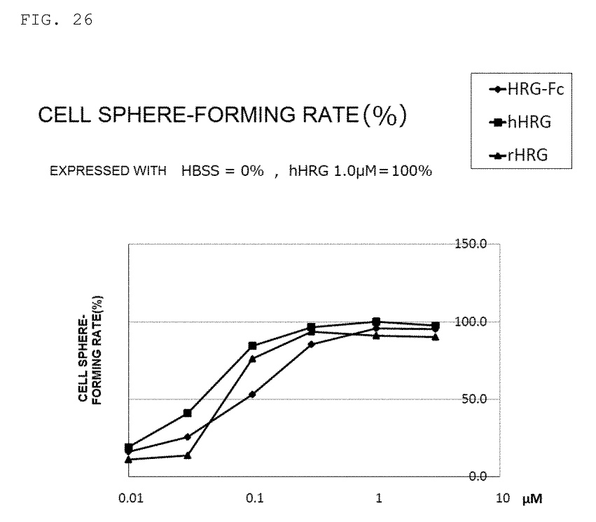

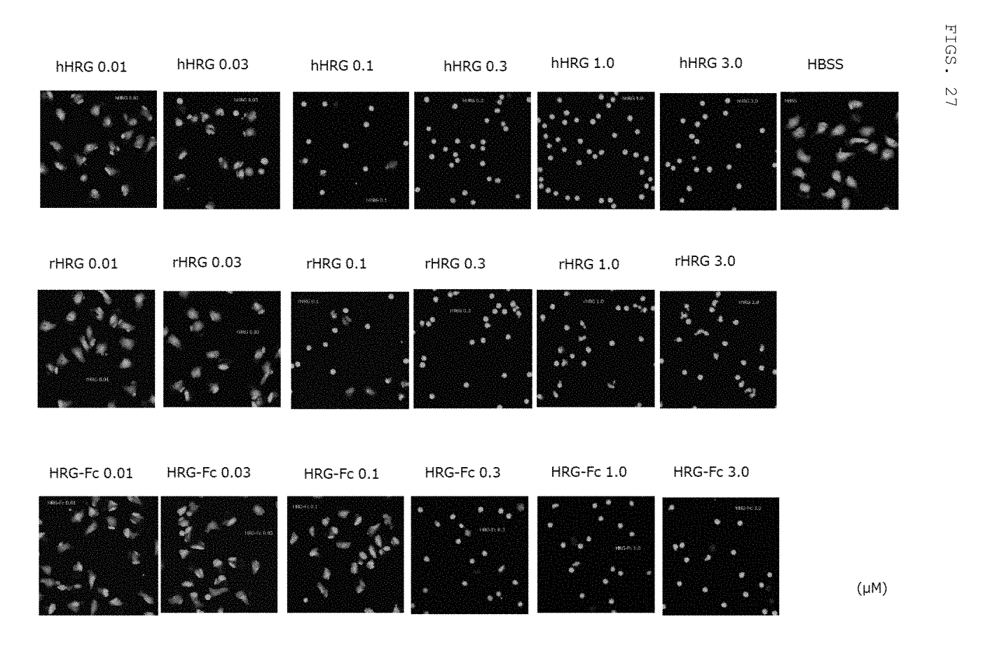

The neutrophil suspension was dispensed into a 96 well plate at 100 μl per well, and the rHRG, the HRG-Fc, and the hHRG were each added at various concentrations of from 0.01 μM to 3 μM of the HRG, followed by incubation at 37° C. for 60 minutes. After 10 minutes of room-temperature cooling, a cell morphology was photographed under a fluorescence microscope, and the cell surface area ratio of the neutrophils was measured with In Cell Analyzer™ 2000 (GE Healthcare), followed by analysis with In Cell Analyzer Workstation software (n=3). The cell sphere-forming rate (%) of the neutrophils with HRG is shown in FIG. 26, confirming the neutrophil sphere-forming activity of HRG. 100% indicates that the cells have a spherical morphology (round morphology of a perfect circle). FIG. 27 are photographs for showing the shapes of the cells when the cells are treated under the conditions of this Experimental Example.

The results of the foregoing confirmed that the rHRG and the HRG-Fc generated by the methods of Examples of the present invention had nearly equivalent neutrophil sphere-forming activity to that of the plasma-derived HRG (hHRG).

### (Experimental Example 6) Blocking Effect on Cell Chemotaxis-Promoting Action of S100A8/A9

In this Experimental Example, for each of the proteins generated with the NPTN-Fc (Example 6), the EMMPRIN-Fc (Example 5), the RAGE-Fc (Example 8), the ALCAM-Fc (Example 10), the MCAM-Fc (Example 9), and the EMB-Fc (Example 7), a blocking effect on the cancer cell chemotaxis-promoting action of S100A8/A9 was confirmed.

S100A8/A9 is known as a protein that promotes the migratory properties and invasive properties of cancer cells. Of S100A8/A9, an S100A8 gene is identified by Ensembl Gene ID: ENSG00000143546, and an S100A9 gene is identified by Ensembl Gene ID: ENSG00000163220. The test of this Experimental Example was performed using S100A8/A9 proteins generated by a method described in Biochemistry and Biophysics Reports 6 (2016) 94-100 on the basis of the above-mentioned gene information.

In this Experimental Example, the chemotaxis of B16-BL6 cells (murine malignant melanoma cell line) was measured by a Boyden chamber method. The test by the Boyden chamber method in this Experimental Example was performed using a combination of chambers with a polycarbonate membrane having a pore size of from 3 μm to 12 μm and a 24 well plate. The lower chamber was filled with 800 μl of DMEM/F12 medium (containing 0.1% serum) containing S100A8/A9 at a final concentration of 100 ng/ml. After that, a cell suspension containing B16-BL6 cells at 5×10⁴ cells/200 μl in DMEM/F12 serum-free medium was added to the upper chamber, and incubated at 37° C. for 12 hours, and then cells that had passed through the chamber membrane were counted.

The proteins generated with the NPTN-Fc (Example 6), the EMMPRIN-Fc (Example 5), the RAGE-Fc (Example 8), the ALCAM-Fc (Example 10), the MCAM-Fc (Example 9), and the EMB-Fc (Example 7) were each added at a final concentration of 1,000 ng/ml to DMEM/F12 medium (containing 0.1% serum) containing S100A8/A9 to prepare each test solution. The lower chamber was filled with 800 μl of each test solution, and then as described above, a cell suspension containing B16-BL6 cells at 5×10⁴ cells/200 μl in DMEM/F12 serum-free medium was added to the upper chamber, and incubated at 37° C. for 12 hours. In each case, cells that had passed through the chamber membrane were counted, and the chemotaxis of the cells was measured. For each of the proteins generated in Examples, a blocking effect on the cell chemotaxis-promoting action of S100A8/A9 was confirmed.

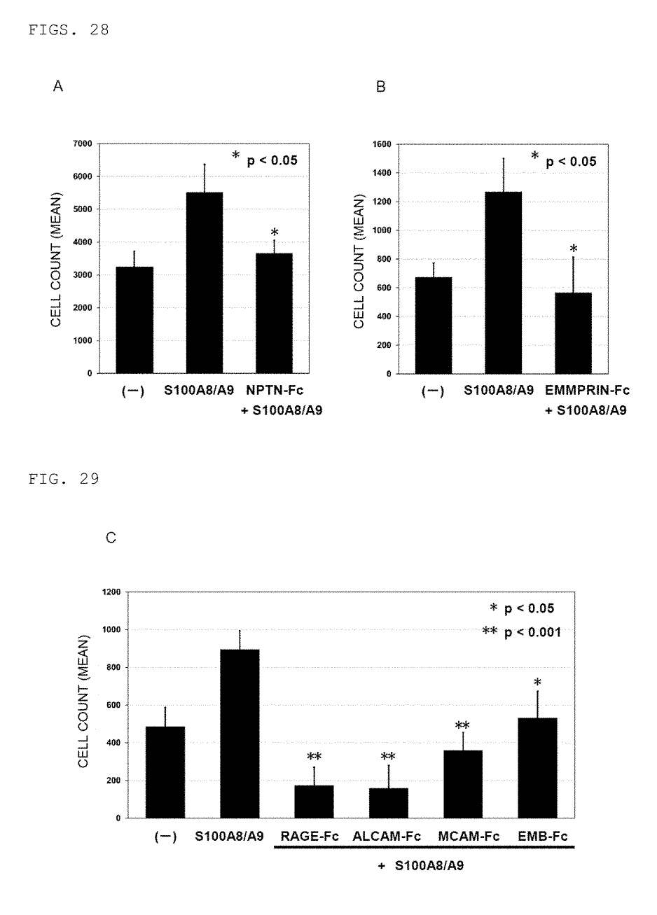

The results confirmed that each of the proteins generated in Examples blocked the cell chemotaxis-promoting action of S100A8/A9, and suppressed the chemotaxis to a level equivalent to or lower than chemotaxis in the case of containing no S100A8/A9 (FIG. 28 and FIG. 29). From the results, it was able to be confirmed that each of the proteins generated by the method of Examples of the present invention had an excellent action.

### INDUSTRIAL APPLICABILITY

As described in detail above, according to the gene expression cassette having a structure in which the DNA construct (X) containing a gene of interest and a poly A addition sequence is sandwiched between the promoter (P) and the enhancer (P′), the gene expression cassette further including the transposon sequences (T) upstream of the promoter (P) and downstream of the enhancer (P′), even a protein that has hitherto been difficult to generate by gene recombination can be produced in a large amount. Further, in the above-mentioned gene expression cassette, when the nuclear matrix binding sequence (M) is appropriately arranged upstream of the replication initiation sequence (S) in combination with the transposon sequence (T), the protein of interest can be more effectively produced stably and in a large amount.

According to the present invention, a gene expression cassette suited for transient expression (e.g., a pCMViR-TSC vector described in Patent Literature 4) is sandwiched between the transposon sequences (T), and thus a large number of copies of the gene expression cassette can be inserted into chromosomes with high efficiency. When the replication initiation sequence (S) and the nuclear matrix binding sequence (M) are further linked upstream or downstream, or upstream and downstream of the gene expression cassette, the number of copies of the gene expression cassette can be amplified with high efficiency. Specifically, cells that stably and highly produce the protein of interest are obtained by linking: the transposon sequence (T) upstream of the promoter (P); the DNA construct (X) containing a gene of interest and a poly A addition sequence downstream of the promoter (P); the enhancer (P′) downstream of the DNA construct (X); and the nuclear matrix binding sequence (M), the replication initiation sequence (S), and the transposon sequence (T) downstream of the enhancer (P′). According to the gene expression vector of the present invention, there has been achieved an expression amount surpassing even the transient expression amount of the pCMViR-TSC vector, which has achieved expression several times to several tens times as high as that achieved by a related-art expression vector, even in a stably expressing cell line after drug selection.

For example, irrespective of the kind of cells, the kind of gene, and the kind of transfection reagent, the protein of interest to be expressed from the gene can be produced stably and in a large amount by ultra-high expression. Such protein can be not only applied as a reagent in the field of biotechnology, but also applied as a therapeutic protein pharmaceutical and widely applied for clinical therapy/examination/diagnosis using a gene.

Specific examples of the protein that may be used for research and development in a medical field, a pharmaceutical, a pharmaceutical product, a diagnostic drug, or a reagent include HRG, PD-1, EMMPRIN, NPTNβ, EMB, RAGE, MCAM, ALCAM, ErbB2, and an antibody.

1-13. (canceled)

14. A gene expression cassette having a structure in which a DNA construct (X) containing a gene of interest and a poly A addition sequence is sandwiched between a promoter (P) and an enhancer (P′), the gene expression cassette further comprising transposon sequences (T) upstream of the promoter (P) and downstream of the enhancer (P′).

15. The gene expression cassette according to claim 14, further comprising:
a replication initiation sequence (S), which is arranged upstream of the promoter (P) and/or downstream of the enhancer (P′); and
a nuclear matrix binding sequence (M), which is arranged upstream of the replication initiation sequence (S).

16. The gene expression cassette according to claim 15, wherein the gene expression cassette comprises the promoter (P), the DNA construct (X) containing a gene of interest and a poly A addition sequence, the enhancer (P′), and the transposon sequences (T), and optionally further comprises the replication initiation sequence (S) in any one of the following orders 1) to 3):
1) (T), (M), (S), (P), (X), (P′), (T);
2) (T), (P), (X), (P′), (M), (S), (T); and
3) (T), (M), (S), (P), (X), (P′), (M), (S), (T).

17. A gene expression cassette, comprising:
a transposon sequence (T);
a promoter (P);
a DNA construct (X) containing a gene of interest and a poly A addition sequence;
an enhancer (P′);
a nuclear matrix binding sequence (M);
a replication initiation sequence (S); and
another transposon sequence (T),
wherein the components (T), (P), (X), (P′), (M), (S), and (T) are arranged in the stated order, and
wherein host cells for the gene expression cassette comprise CHO cells.

18. The gene expression cassette according to claim 15, wherein the replication initiation sequence (S) comprises ROIS and/or ARS.

19. The gene expression cassette according to claim 15, wherein the promoter (P) comprises a promoter selected from the group consisting of a CMV promoter, a CMV-i promoter, an SV40 promoter, an hTERT promoter, a β-actin promoter, and a CAG promoter.

20. The gene expression cassette according to claim 15, wherein the enhancer (P′), which is linked downstream of the DNA construct (X) containing a gene of interest and a poly A addition sequence, contains any one kind or a plurality of kinds selected from an hTERT enhancer, a CMV enhancer, and an SV40 enhancer.

21. The gene expression cassette according to claim 15, wherein, in the DNA construct (X) containing a gene of interest and a poly A addition sequence, the gene of interest contains a gene encoding part or a whole of a protein selected from HRG, PD-1, EMMPRIN, NPTNβ, EMB, RAGE, MCAM, ALCAM, ErbB2, and an antibody.

22. The gene expression plasmid, comprising the gene expression cassette of claim 15.

23. The gene expression vector, comprising the gene expression cassette of claim 15.

24. A method of expressing a gene of interest, comprising using the expression cassette of claim 15.

25. A protein, which is produced using the gene expression cassette of claim 15.

26. The gene expression cassette according to claim 17, wherein the replication initiation sequence (S) comprises ROIS and/or ARS.

27. The gene expression cassette according to claim 17, wherein the promoter (P) comprises a promoter selected from the group consisting of a CMV promoter, a CMV-i promoter, an SV40 promoter, an hTERT promoter, a β-actin promoter, and a CAG promoter.

28. The gene expression cassette according to claim 17, wherein the enhancer (P′), which is linked downstream of the DNA construct (X) containing a gene of interest and a poly A addition sequence, contains any one kind or a plurality of kinds selected from an hTERT enhancer, a CMV enhancer, and an SV40 enhancer.

29. The gene expression cassette according to claim 17, wherein, in the DNA construct (X) containing a gene of interest and a poly A addition sequence, the gene of interest contains a gene encoding part or a whole of a protein selected from HRG, PD-1, EMMPRIN, NPTNβ, EMB, RAGE, MCAM, ALCAM, ErbB2, and an antibody.

30. The gene expression plasmid, comprising the gene expression cassette of claim 17.

31. The gene expression vector, comprising the gene expression cassette of claim 17.

32. A method of expressing a gene of interest, comprising using the expression cassette of claim 17.

33. A protein, which is produced using the gene expression cassette of claim 17.


SEQUENCE LISTING (29 sequences)

SEQ ID NO: 1 - DNA, 604 bp, Homo sapiens
ctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggcc

SEQ ID NO: 2 - DNA, 634 bp, Artificial
  Whole sequence of 5′TP with Mlu1-AflII-5′ TP-Sal1
acgcgtaaacttcttaagaatctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccaatgtcgac

SEQ ID NO: 3 - DNA, 1090 bp, Homo sapiens
ttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggata

SEQ ID NO: 4 - DNA, 1122 bp, Artificial
  Whole sequence of 3′TP with Kpn1-3′ TP-BssHII-Not1
ggtaccccgttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggatattggcgcgccaactggcggccgc

SEQ ID NO: 5 - DNA, 1050 bp, Homo sapiens
aatctgagccaagtagaagaccttttcccctcctacccctactttctaagtcacagaggctttttgttcccccagacactcttgcagattagtccaggcagaaacagttagatgtccccagttaacctcctatttgacaccactgattaccccattgatagtcacactttgggttgtaagtgactttttatttatttgtatttttgactgcattaagaggtctctagttttttacctcttgtttcccaaaacctaataagtaactaatgcacagagcacattgatttgtatttattctatttttagacataatttattagcatgcatgagcaaattaagaaaaacaacaacaaatgaatgcatatatatgtatatgtatgtgtgtacatatacacatatatatatatattttttttcttttcttaccagaaggttttaatccaaataaggagaagatatgcttagaactgaggtagagttttcatccattctgtcctgtaagtattttgcatattctggagacgcaggaagagatccatctacatatcccaaagctgaattatggtagacaaagctcttccacttttagtgcatcaatttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcacctgactcctgaggagaagtctgccgttactgccctgtggggcaaggtgaacgtg

SEQ ID NO: 6 - DNA, 456 bp, Homo sapiens
tagcttgtattttttgtaatttaaaataatgatgtattaaaaacatttgtattctctatatatattttaaatttagtttaatttcataaacatttctcaagagtatattttgtgcagggcatattgctagtcattatgggatctatatagttatgttaaatttaaagtatggtcttacgggggaagatgatagaaaatgtacatttataaacttcctgcaatgtatgagttattatgttataaacttttacatattttgacccatttaatccccattttgtagatgagtagactgaggctcatgaaatgataaagattttcccatggtatcaggaataagagttgtcaaagtaaaattaaaaccaggacttttggctccctaaagctattctaatgctattatttcaagcataaaggctagtttttatgtaagttataaaagagatacacatttac

SEQ ID NO: 7 - DNA, 368 bp, Homo sapiens
taccacacagtctaagctgaacctggttggttaacttgaaaaatgcagagatgtagttacatcagcagtgggaagacaagaagatcagtttcagtgggagaagtcattgcattgggaggggtaattaacagagtggtagcatatgtggaatgtgggctctatagataaggactggcaggaatgttgtgtaccagggctggggggatatagagggtaaggaagtctggccttgaaatcagggaacaaaggacaacaaaacttaaacgagctaaacctttgaagaagaatttcttactgtagtcagcgatcattattgtaaacctatgacagttctttcaaaatatttttcagacttgtcaaccgctgta

SEQ ID NO: 8 - DNA, 6088 bp, Artificial
  Gene Expression Cassette No.4
acgcgtaaacttcttaagaatctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccaatgtcgacgtcggccataaattttttgcaaaagccttggcctccaaaaaagcctcctcactacttctggaatagctcagaggccgaggcggcctcggcctctgcataaataaaaaaaattagtcagccttggggcggagaaactatcgttgctgactaattgagatcggagtactgtcctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatccatcaattaatacgactcactatagggagacagactgttcctttcctgggtcttttctggcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagccaccccggaattccggggactagtcccgcggatccgcgcccaagcttgggccgctcgagcgggctctagagctagatgactaacgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggagtactgtcctccgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagttttggaaagtccccaggctccccagcaggcagaagtatccaaagcatccatctcaattagtcagcaaccaggtgtggaaagtccccaggctccccagcaggcagaagtatccaaagcatccatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggccaaggcttttgcaaaaagctccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgctagctagctagatgactaacgtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcatcttggaccattagctccacaggtatcttcttccctctagtggtcataacagcagcttcagctacctctcgggtttaaaccctaccacacagtctaagctgaacctggttggttaacttgaaaaatgcagagatgtagttacatcagcagtgggaagacaagaagatcagtttcagtgggagaagtcattgcattgggaggggtaattaacagagtggtagcatatgtggaatgtgggctctatagataaggactggcaggaatgttgtgtaccagggctggggggatatagagggtaaggaagtctggccttgaaatcagggaacaaaggacaacaaaacttaaacgagctaaacctttgaagaagaatttcttactgtagtcagcgatcattattgtaaacctatgacagttctttcaaaatatttttcagacttgtcaaccgctgtaaatctgagccaagtagaagaccttttcccctcctacccctactttctaagtcacagaggctttttgttcccccagacactcttgcagattagtccaggcagaaacagttagatgtccccagttaacctcctatttgacaccactgattaccccattgatagtcacactttgggttgtaagtgactttttatttatttgtatttttgactgcattaagaggtctctagttttttacctcttgtttcccaaaacctaataagtaactaatgcacagagcacattgatttgtatttattctatttttagacataatttattagcatgcatgagcaaattaagaaaaacaacaacaaatgaatgcatatatatgtatatgtatgtgtgtacatatacacatatatatatatattttttttcttttcttaccagaaggttttaatccaaataaggagaagatatgcttagaactgaggtagagttttcatccattctgtcctgtaagtattttgcatattctggagacgcaggaagagatccatctacatatcccaaagctgaattatggtagacaaagctcttccacttttagtgcatcaatttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcacctgactcctgaggagaagtctgccgttactgccctgtggggcaaggtgaacgtgccttaattaaggcggggtaccccgttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggatattggcgcgccaactggcggccgc

SEQ ID NO: 9 - DNA, 4303 bp, Artificial
  Gene Expression Cassette No.1
acgcgtaaacttcttaagaatctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccaatgtcgacgtcggccataaattttttgcaaaagccttggcctccaaaaaagcctcctcactacttctggaatagctcagaggccgaggcggcctcggcctctgcataaataaaaaaaattagtcagccttggggcggagaaactatcgttgctgactaattgagatcggagtactgtcctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatccatcaattaatacgactcactatagggagacagactgttcctttcctgggtcttttctggcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagccaccccggaattccggggactagtcccgcggatccgcgcccaagcttgggccgctcgagcgggctctagagctagatgactaacgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggagtactgtcctccgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagttttggaaagtccccaggctccccagcaggcagaagtatccaaagcatccatctcaattagtcagcaaccaggtgtggaaagtccccaggctccccagcaggcagaagtatccaaagcatccatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggccaaggcttttgcaaaaagctccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgcggggtaccccgttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggatattggcgcgccaactggcggccgc

SEQ ID NO: 10 - protein, 507 aa, Homo sapiens
VSPTDCSAVEPEAEKALDLINKRRRDGYLFQLLRIADAHLDRVENTTVYYLVLDVQESDCSVLSRKYWNDCEPPDSRRPSEIVIGQCKVIATRHSHESQDLRVIDFNCTTSSVSSALANTKDSPVLIDFFEDTERYRKQANKALEKYKEENDDFASFRVDRIERVARVRGGEGTGYFVDFSVRNCPRHHFPRHPNVFGFCRADLFYDVEALDLESPKNLVINCEVFDPQEHENINGVPPHLGHPFHWGGHERSSTTKPPFKPHGSRDHHHPHKPHEHGPPPPPDERDHSHGPPLPQGPPPLLPMSCSSCQHATFGTNGAQRHSHNNNSSDLHPHKHHSHEQHPHGHHPHAHHPHEHDTHRQHPHGHHPHGHHPHGHHPHGHHPHGHHPHCHDFQDYGPCDPPPHNQGHCCHGHGPPPGHLRRRGPGKGPRPFHCRQIGSVYRLPPLRKGEVLPLPEANFPSFPLPHHKHPLKPDNQPFPQSVSESCPGKFKSGFPQVSMFFTHTFPK

SEQ ID NO: 11 - DNA, 722 bp, Artificial
  XhoI-GPG-hIgG2 Fc-Xba1
ctcgagcggggacctggagagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaagggctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgactaactagcgctctaga

SEQ ID NO: 12 - protein, 227 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 13 - DNA, 531 bp, Artificial
  EcoR1-exPD-1-Xho1
gaattccggaccatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtgccgctcgag

SEQ ID NO: 14 - protein, 149 aa, Homo sapiens
GWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 15 - DNA, 639 bp, Artificial
  EcoR1-exEMMPRIN-Xho1
gaattccggaccatggcggctgcgctgttcgtgctgctgggattcgcgctgctgggcacccacggagcctccggggctgccggcacagtcttcactaccgtagaagaccttggctccaagatactcctcacctgctccttgaatgacagcgccacagaggtcacagggcaccgctggctgaaggggggcgtggtgctgaaggaggacgcgctgcccggccagaaaacggagttcaaggtggactccgacgaccagtggggagagtactcctgcgtcttcctccccgagcccatgggcacggccaacatccagctccacgggcctcccagagtgaaggctgtgaagtcgtcagaacacatcaacgagggggagacggccatgctggtctgcaagtcagagtccgtgccacctgtcactgactgggcctggtacaagatcactgactctgaggacaaggccctcatgaacggctccgagagcaggttcttcgtgagttcctcgcagggccggtcagagctacacattgagaacctgaacatggaggccgaccccggccagtaccggtgcaacggcaccagctccaagggctccgaccaggccatcatcacgctccgcgtgcgcagccacctgccgctcgag

SEQ ID NO: 16 - protein, 188 aa, Homo sapiens
ASGAAGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHL

SEQ ID NO: 17 - DNA, 1035 bp, Artificial
  EcoR1-exNPTN-Xho1
gaattccggaccatggggtcgggttcgtcgctgcccagcgccctggccctctcgctgttgctggtctctggctccctcctcccagggccaggcgccgctcagaacgctgggtttgtcaagtcgcccatgtcagaaactaagctcacgggggacgcctttgagctgtactgtgacgtggtcgggagccccacgccagagatccagtggtggtacgcagaagtcaaccgggcagagtctttcagacagctgtgggacggtgctcggaagcgccgtgtcaccgtaaacaccgcctacgggtcaaacggcgtgagtgtgctgagaataacccggctcaccttggaggactctgggacttacgagtgcagggccagcaacgaccccaagaggaatgacttgaggcaaaacccctccataacatggattcgagcccaggccaccataagcgtccttcagaagccaaggattgtcaccagtgaagaggtcattattcgagacagccctgttctccctgtcaccctgcagtgtaacctcacctccagctctcacacccttacatacagctactggacaaagaatggggtggaactgagtgccactcgtaagaatgccagcaacatggagtacaggatcaataagccgagagctgaggattcaggcgaataccactgcgtatatcactttgtcagcgctcctaaagcaaacgccaccattgaagtgaaagccgctcctgacatcactggccataaacggagtgagaacaagaatgaagggcaggatgccactatgtattgcaagtcagttggctacccccacccagactggatatggcgcaagaaggagaacgggatgcccatggacattgtcaatacctctggccgcttcttcatcatcaacaaggaaaattacactgagttgaacattgtgaacctgcagatcacggaagaccctggcgagtatgaatgtaatgccaccaacgccattggctccgcctctgttgtcactgtcctcagggtgcggagccacctgccgctcgag

SEQ ID NO: 18 - protein, 309 aa, Homo sapiens
QNAGFVKSPMSETKLTGDAFELYCDVVGSPTPEIQWWYAEVNRAESFRQLWDGARKRRVTVNTAYGSNGVSVLRITRLTLEDSGTYECRASNDPKRNDLRQNPSITWIRAQATISVLQKPRIVTSEEVIIRDSPVLPVTLQCNLTSSSHTLTYSYWTKNGVELSATRKNASNMEYRINKPRAEDSGEYHCVYHFVSAPKANATIEVKAAPDITGHKRSENKNEGQDATMYCKSVGYPHPDWIWRKKENGMPMDIVNTSGRFFIINKENYTELNIVNLQITEDPGEYECNATNAIGSASVVTVLRVRSHL

SEQ ID NO: 19 - DNA, 798 bp, Artificial
  EcoR1-exEMB-Xho1
gaattccggatgcgcgccctccccggcctgctggaggccagggcgcgtacgccccggctgctcctcctccagtgccttctcgctgccgcgcgcccaagctcggcggacggcagtgccccagattcgccttttacaagtccacctctcagagaagaaataatggcaaataacttttccttggagagtcataacatatcactgactgaacattctagtatgccagtagaaaaaaatatcactttagaaaggccttctaatgtaaatctcacatgccagttcacaacatctggggatttgaatgcagtaaatgtgacttggaaaaaagatggtgaacaacttgagaataattatcttgtcagtgcaacaggaagcaccttgtatacccaatacaggttcaccatcattaatagcaaacaaatgggaagttattcttgtttctttcgagaggaaaaggaacaaaggggaacatttaatttcaaagtccctgaacttcatgggaaaaacaagccattgatctcttacgtaggggattctactgtcttgacatgtaaatgtcaaaattgttttcctttaaattggacctggtacagtagtaatgggagtgtaaaggttcctgttggtgttcaaatgaataaatatgtgatcaatggaacatatgctaacgaaacaaagctgaagataacacaacttttggaggaagatggggaatcttactggtgccgtgcactattccaattaggcgagagtgaagaacacattgagcttgtggtgctgagctatttggtgcccccgctcgag

SEQ ID NO: 20 - protein, 228 aa, Homo sapiens
DGSAPDSPFTSPPLREEIMANNFSLESHNISLTEHSSMPVEKNITLERPSNVNLTCQFTTSGDLNAVNVTWKKDGEQLENNYLVSATGSTLYTQYRFTIINSKQMGSYSCFFREEKEQRGTFNFKVPELHGKNKPLISYVGDSTVLTCKCQNCFPLNWTWYSSNGSVKVPVGVQMNKYVINGTYANETKLKITQLLEEDGESYWCRALFQLGESEEHIELVVLSYLVP

SEQ ID NO: 21 - DNA, 1032 bp, Artificial
  EcoR1-exRAGE-Xho1
gaattccggaccatggctgccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggccgctcgag

SEQ ID NO: 22 - protein, 319 aa, Homo sapiens
QNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLA

SEQ ID NO: 23 - DNA, 1698 bp, Artificial
  EcoR1-exMCAM-BamH1
gaattccggaccatggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccagtcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctgccggagccggagagccggggccgcggatcc

SEQ ID NO: 24 - protein, 536 aa, Homo sapiens
VPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQGLDLDTMISLLSEPQELLVNYVSDVRVSPAAPERQEGSSLTLTCEAESSQDLEFQWLREETGQVLERGPVLQLHDLKREAGGGYRCVASVPSIPGLNRTQLVNVAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESRG

SEQ ID NO: 25 - DNA, 1602 bp, Artificial
  EcoR1-exALCAM-BamH1
gaattccggaccatggaatccaagggggccagttcctgccgtctgctcttctgcctcttgatctccgccaccgtcttcaggccaggccttggatggtatactgtaaattcagcatatggagataccattatcataccttgccgacttgacgtacctcagaatctcatgtttggcaaatggaaatatgaaaagcccgatggctccccagtatttattgccttcagatcctctacaaagaaaagtgtgcagtacgacgatgtaccagaatacaaagacagattgaacctctcagaaaactacactttgtctatcagtaatgcaaggatcagtgatgaaaagagatttgtgtgcatgctagtaactgaggacaacgtgtttgaggcacctacaatagtcaaggtgttcaagcaaccatctaaacctgaaattgtaagcaaagcactgtttctcgaaacagagcagctaaaaaagttgggtgactgcatttcagaagacagttatccagatggcaatatcacatggtacaggaatggaaaagtgctacatccccttgaaggagcggtggtcataatttttaaaaaggaaatggacccagtgactcagctctataccatgacttccaccctggagtacaagacaaccaaggctgacatacaaatgccattcacctgctcggtgacatattatggaccatctggccagaaaacaattcattctgaacaggcagtatttgatatttactatcctacagagcaggtgacaatacaagtgctgccaccaaaaaatgccatcaaagaaggggataacatcactcttaaatgcttagggaatggcaaccctcccccagaggaatttttgttttacttaccaggacagcccgaaggaataagaagctcaaatacttacacactaacggatgtgaggcgcaatgcaacaggagactacaagtgttccctgatagacaaaaaaagcatgattgcttcaacagccatcacagttcactatttggatttgtccttaaacccaagtggagaagtgactagacagattggtgatgccctacccgtgtcatgcacaatatctgctagcaggaatgcaactgtggtatggatgaaagataacatcaggcttcgatctagcccgtcattttctagtcttcattatcaggatgctggaaactatgtctgcgaaactgctctgcaggaggttgaaggactaaagaaaagagagtcattgactctcattgtagaaggcaaacctcaaataaaaatgacaaagaaaactgatcccagtggactatctaaaacaataatctgccatgtggaaggttttccaaagccagccattcaatggacaattactggcagtggaagcgtcataaaccaaacagaggaatctccttatattaatggcaggtattatagtaaaattatcatttcccctgaagagaatgttacattaacttgcacagcagaaaaccaactggagagaacagtaaactccttgaatgtctctgctataagtattccagaacacgatgaggcagacgagataagtgatgaaaacagagaaaaggtgaatgaccaggcaaaacgcggatcc

SEQ ID NO: 26 - protein, 500 aa, Homo sapiens
WYTVNSAYGDTIIIPCRLDVPQNLMFGKWKYEKPDGSPVFIAFRSSTKKSVQYDDVPEYKDRLNLSENYTLSISNARISDEKRFVCMLVTEDNVFEAPTIVKVFKQPSKPEIVSKALFLETEQLKKLGDCISEDSYPDGNITWYRNGKVLHPLEGAVVIIFKKEMDPVTQLYTMTSTLEYKTTKADIQMPFTCSVTYYGPSGQKTIHSEQAVFDIYYPTEQVTIQVLPPKNAIKEGDNITLKCLGNGNPPPEEFLFYLPGQPEGIRSSNTYTLTDVRRNATGDYKCSLIDKKSMIASTAITVHYLDLSLNPSGEVTRQIGDALPVSCTISASRNATVVWMKDNIRLRSSPSFSSLHYQDAGNYVCETALQEVEGLKKRESLTLIVEGKPQIKMTKKTDPSGLSKTIICHVEGFPKPAIQWTITGSGSVINQTEESPYINGRYYSKIIISPEENVTLTCTAENQLERTVNSLNVSAISIPEHDEADEISDENREKVNDQAK

SEQ ID NO: 27 - DNA, 1974 bp, Artificial
  HindIII-exErbB2-Xho1
aagcttgggatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggagggctgcgggagctgcagcttcgaagcctcacagagattttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtggggtcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatttttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgccgctcgag

SEQ ID NO: 28 - protein, 630 aa, Homo sapiens
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 29 - DNA, 1596 bp, Artificial
  EcoR1-HRG-Xho1
gaattccggatggggaaggcactcattgcagcactgcttttgatcacattgcagtattcgtgtgccgtgagtcccactgactgcagtgctgttgagccggaggctgagaaagctctagacctgatcaataaaaggcgacgggatggctaccttttccaattgctgcggattgctgatgcccacttggacagagtggaaaatacaactgtatattacttagtcttagatgtgcaagaatcggactgttcggtcctatccaggaaatactggaatgactgtgagccacctgattccagacgtccatctgaaatagtgatcggacaatgtaaggtaatagctacaagacattcccatgaatctcaggacctcagagtgattgactttaactgcaccacaagttctgtctcttcagcactggccaataccaaagatagtccggtcctcatagatttctttgaggatactgagcgctacagaaaacaagccaacaaagcccttgagaagtacaaagaggagaatgatgactttgcctctttcagagtggaccgaatcgagagagttgcaagagtgagaggaggggaaggaactggttacttcgtggacttctctgtgcggaactgccccagacaccatttccccagacaccccaatgtctttggattctgcagagcagatttgttctatgatgtagaagccttggacttggaaagcccgaaaaaccttgtcataaactgtgaagtcttcgaccctcaggaacatgagaacatcaatggtgtaccgcctcatttgggacatcccttccactggggtgggcatgagcgttcttctaccaccaagcctccattcaagccccatggatctagagatcatcatcatccccacaagccacacgaacatggacccccacctcctccagatgaaagagatcactcacatggacccccacttccacaaggccctcctccactattgcccatgtcctgctcaagttgtcaacatgccacttttggcacaaatggggcccaaagacattctcataataataattccagtgacctccatccccataagcatcattcccatgaacagcatccccacggacaccatccccatgcacaccatcctcatgaacatgatacccatagacagcatccccatggacaccacccccatggacaccatcctcatggacaccacccccatggacaccatccccatggacaccatccccactgccatgatttccaagactatggaccttgtgacccaccaccccataaccaaggtcactgttgccatggccacggcccaccacctgggcacttaagaaggcgaggcccaggtaaaggaccccgtcccttccattgcagacaaattggatctgtgtaccgactccctcctctaagaaaaggtgaggtgctgccacttcctgaggccaattttcccagcttcccattgccgcaccacaaacatcctctaaagccagacaatcagccctttcctcaatcagtctctgaatcatgtccagggaagttcaagagtgggtttccacaagtttccatgttttttacacatacatttccaaaaccgctcgag